(19) United States
(12) Patent Application Publication
(10) Pub. No.: US 2010/0331630 A1
(43) Pub. Date: Dec. 30, 2010

(54) MACHINE, MANUFACTURE, AND PROCESS FOR ANALYZING THE RELATIONSHIP BETWEEN DISPOSABLE DIAPER WEAR WITH SLEEP AND/OR DEVELOPMENTAL INDICATORS

(76) Inventor: Mauricio Rolando Odio, Gahanna, OH (US)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/491,274

(22) Filed: Jun. 25, 2009

(51) Int. Cl. A61B 5/00; G09B 19/00

(52) U.S. Cl. 600/301; 434/236

(57) ABSTRACT

A machine or system, article of manufacture or computer program product, or process for facilitating the analysis of the effect of diaper wear on sleep and/or developmental indicators including verifying a test diaper was secured on at least one of a first plurality of subjects; monitoring the subject for sleep initiation data; evaluating the sleep of the subject based upon sleep evaluation data.

### FIELD OF THE INVENTION

The present technical disclosure relates to methods and products for analyzing the relationship between diaper wear with sleep and/or developmental indicators.

### BACKGROUND OF THE INVENTION

Conventional systems analyze and/or evaluate the relation between sleep and cognition. For example, in a normative sample, the amount of sleep obtained at night by American infants was negatively related to mood, adaptability, rhythmicity, and approach. Similar findings resulted from a cross-cultural comparison of Chinese-American and non-Chinese infants.

In particular, the Weissbluth (1982) study asked parents to rate the temperament of their infants on the Carey Infant Temperament Questionnaire and report on the nighttime sleep habits of their infants. The parents of Chinese-American infants rated the temperament of their infants as more difficult relative to non-Chinese infants, such that they were more withdrawn, less adaptable, and more negative in mood. Notably, Chinese-American infants also slept less at night relative to non-Chinese infants. These findings are similar to those obtained from infants and children with sleep problems, which indicate that individuals with difficult temperament profiles (characterized by difficulty approaching and adapting to novel situations as well as intense negative reactions) are more likely to experience nighttime sleep problems relative to those with easy temperaments (characterized by easy approach and adaptation to the new as well as mild, positive mannerisms). Taken together, these data indicate that sleep and temperament are related.

However, because none of the published studies has included experimental manipulation of nighttime sleep habits, the work does not indicate whether sleep problems cause infants to develop difficult temperaments, or whether having a difficult temperament predisposes infants to sleep problems.

In other words, conventional analysis established associations between sleep habits and temperament in infancy and early childhood. However the conventional analysis has not included experimental manipulations designed to evaluate causal relations of diaper wear with sleep and/or developmental indicators.

### SUMMARY OF THE INVENTION

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

Embodiments disclosed herein include an analysis machine configured to facilitate analysis of the effect of diaper wear on sleep and/or developmental indicators comprising a disposable diaper verification module configured to accept verification input indicating that a disposable diaper was secured on at least one of a first plurality of subjects; a sleep initiation monitoring module configured to monitor the subject for sleep initiation data; a sleep evaluation module configured to evaluate the sleep of the subject based upon sleep evaluation data.

Embodiments disclosed herein include an analysis machine configured to facilitate analysis of the effect of diaper wear on developmental indicators comprising: a disposable diaper verification module configured to accept verification input indicating that a disposable diaper was secured on at least one of a first plurality of subjects; a sleep initiation monitoring module configured to monitor the subject for sleep initiation data; a sleep evaluation module configured to evaluate the sleep of the subject based upon sleep evaluation data; a developmental indicator input module configured to accept data of a developmental indicator of the subject of the first plurality, and a display module configured to display the developmental indicator data or the sleep evaluation data of the subject of the first plurality, a cloth diaper verification module configured to accept verification input indicating that a cloth diaper was secured to at least one of a second plurality of subjects; a sleep initiation monitoring module configured to monitor the subject of the second plurality for sleep initiation data; a sleep evaluation module configured to evaluate the sleep of the subject of the second plurality based upon sleep evaluation data; a developmental indicator input module configured to accept data of a developmental indicator of the subject of the second plurality, and a display module configured to display the developmental indicator data or the sleep evaluation data of the subject of the second plurality, wherein the developmental indicator data and the sleep evaluation data of the subject of the first plurality are collected during a coinciding time duration, wherein the developmental indicator data and the sleep evaluation data of the subject of the second plurality are collected during a coinciding time duration; a comparator module configured to compare the developmental indicator data of the subject of the first plurality and the developmental indicator data of the subject of the second plurality; a comparator module configured to compare data of the sleep of the subject of the first plurality and the sleep of the subject of the second plurality, wherein the sleep initiation monitoring module is any one or more of a group consisting of: a polysomnography device, an actigraphy device, or a video monitoring device, wherein the actigraphy module is any one or more of the grouping consisting of: a zero crossing computing device, a proportional integral computing device, an area under the curve computing device, a time above threshold computing device, or a length of time that the wave is above a certain threshold computing device.

Embodiments disclosed herein include a computer program product having a computer readable medium including programmed instructions for facilitating analysis of the effect of diaper wear on sleep and/or developmental indicators, wherein the instructions, when executed by a processor configured to facilitate the analysis of the effect of diaper wear on the sleep and/or the developmental indicators causes the processor to perform: verifying a disposable diaper was secured on at least one of a first plurality of subjects; monitoring the subject for sleep initiation data; evaluating the sleep of the subject based upon sleep evaluation data.

### BRIEF DESCRIPTION OF THE DRAWINGS

FIG. 1 is an illustration of an embodiment of a machine or manufacture used to analyze the relationship between disposable diaper wear with sleep and/or developmental indicators.

FIG. 2 is an embodiment of an analysis system configured to analyze the relationship between disposable diaper wear with sleep and/or developmental indicators.

FIG. 3 is an embodiment of a process for analyzing the relationship between disposable diaper wear with sleep and/or developmental indicators.

FIG. 4A is an embodiment of data collected and presented by a user interface of a sleep evaluation module which depicts sleep data of a child that experienced a night with prevalent uninterrupted sleep.

FIG. 4B is an embodiment of data collected and presented by a user interface of a sleep evaluation module which depicts sleep data of a child that experienced multiple arousals and disruptions of sleep.

### DETAILED DESCRIPTION OF THE INVENTION

Although the following text sets forth a detailed description of numerous different embodiments, it should be understood that the legal scope of the description is defined by the words of the claims set forth at the end of this patent. The detailed description is to be construed as exemplary only and does not describe every possible embodiment since describing every possible embodiment would be impractical, if not impossible. Numerous alternative embodiments could be implemented, using either current technology or technology developed after the filing date of this patent, which would still fall within the scope of the claims defining the technical disclosure.

It should also be understood that, unless a term is expressly defined in this patent using the sentence “As used herein, the term ‘______’ is hereby defined to mean . . . ” or a similar sentence, there is no intent to limit the meaning of that term, either expressly or by implication, beyond its plain or ordinary meaning, and such term should not be interpreted to be limited in scope based on any statement made in any section of this patent (other than the language of the claims). To the extent that any term recited in the claims at the end of this patent is referred to in this patent in a manner consistent with a single meaning, that is done for sake of clarity only so as to not confuse the reader, and it is not intended that such claim term by limited, by implication or otherwise, to that single meaning.

The present technical disclosure is broadly drawn to embodiments that analyze the relationship between disposable diaper wear with sleep and/or developmental indicators. One embodiment evaluates the relationship between disposable diaper wear with sleep. Another embodiment evaluates the relationship between disposable diaper wear with developmental indicators based on the sleep effects of the diaper wear. Additional embodiments of the technical disclosure are disclosed. Embodiments disclosed include a machine, an article of manufacture, and a process that implements specific purpose machines to analyze the relationship between disposable diaper wear with sleep and/or developmental indicators.

Other embodiments further include a comparison between the resultant sleep data and/or developmental indicator data of subjects that wore diapers which are manufactured to be disposable and the resultant sleep data and/or developmental indicator data of subjects that wore diapers mostly composed of cloth.

Some of the features of the disclosed machine, manufacture, and machine implemented process may be semi-automatically implemented, wherein a human user may provide some of the actions, analysis and/or inputs/outputs to the method. While the technical disclosure is described herein with respect to automatic and semi-automatic systems and methods, it is contemplated that the technical disclosure encompasses systems and methods that manually analyze the relationship between disposable diaper wear with sleep and/or developmental indicators, wherein a human user conducts the analysis.

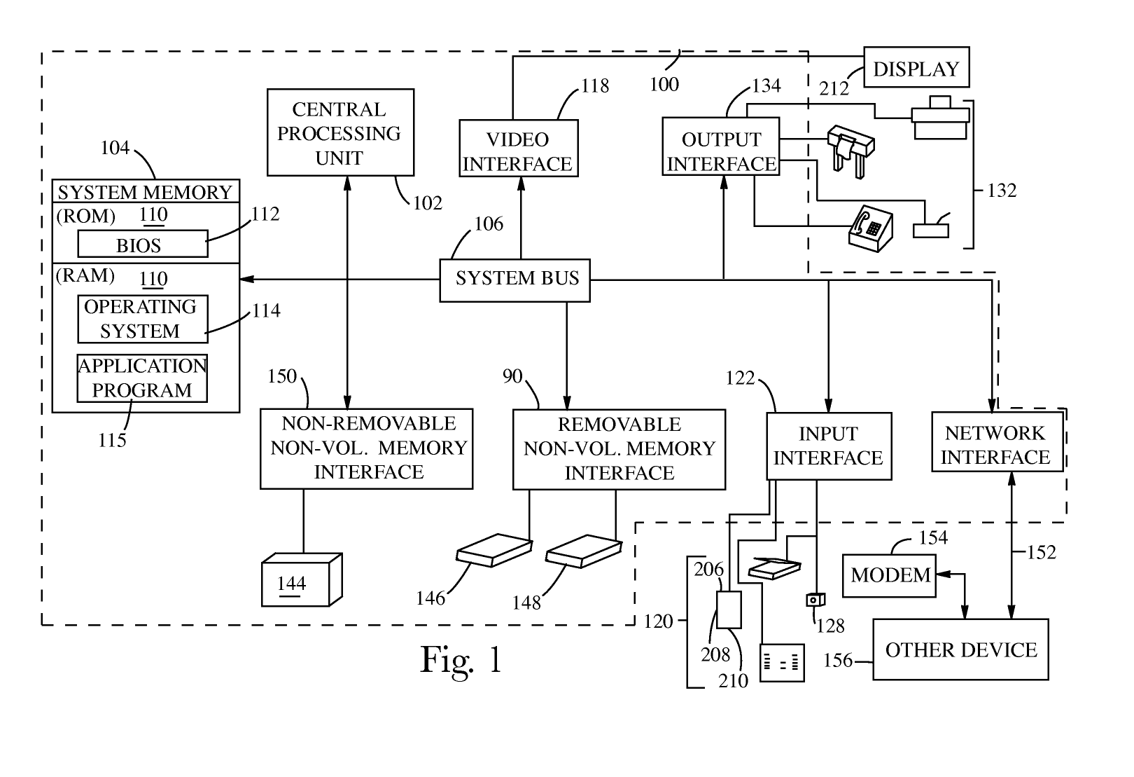

Referring to FIG. 1, a computer system 100 for use with an embodiment of the present disclosure is illustrated. The computer system 100 includes a microprocessor (“CPU”) 102, for example, an Intel Pentium™ class microprocessor. One or more memory devices 102, 144, 146, 148 may be connected to the bus 106, including random access memory (“RAM”) 108, read only memory (“ROM”) 110, a (BIOS) memory 112, video memory, etc. Input and output devices can also be connected to the computer system 100. The input 120 and output 132 devices may include a keyboard, a mouse, a display, a camera or other image capture device, a printer, a plotter, a scanner, a facsimile device, a photocopier, and the like or a device that can be used for both input and output, such as a display, i.e., a touch screen device.

In some embodiments, the computer system 100 includes a computer-readable medium having a computer program or computer system software accessible therefrom, the computer program including instructions for performing the process of the present disclosure. The computer-readable medium may typically include any of the following: a magnetic storage medium, including disk medium, tape storage medium, microdrives, compact flash cards; an optical storage medium, including compact disks such as CD-ROM, CD-RW, and DVD; a non-volatile memory storage medium; a volatile memory storage medium; and data transmission or communications medium including packets of electronic data, and electromagnetic or fiber optic waves modulated in accordance with the instructions. Thus, the computer readable medium tangibly embodies a program, functions, and/or instructions that are executable by the computer system 100 to perform the process of the present technical disclosure disclosure substantially as described herein.

The computer system 100 may be connected to a network, including local area networks (LANs), wide area networks (WANs), portions of the Internet such as a private Internet, a secure Internet, a value-added network, or a virtual private network. Suitable network internal clients include, without limitation, personal computers, laptops, workstations, disconnectable mobile computers, mainframes, information appliances, personal digital assistants, and other handheld and/or embedded processing systems. The signal lines which support communications links to the servers may include twisted pair, coaxial, or optical fiber cables, telephone lines, satellites, microwave relays, modulated AC power lines, and other data transmission “wires” known to those of skill in the art. Although particular individual and network computer systems and components are shown, those of skill in the art will appreciate that the present technical disclosure also works with a variety of other networks and computers, and devices.

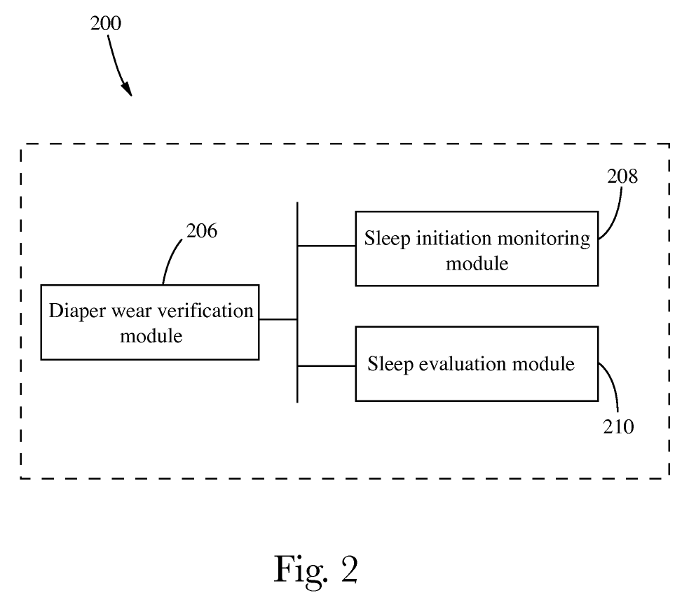

While a wide variety of computer systems are suitable for use with the present machine, article of manufacture, and process embodiments of the technical disclosure, computer system 100 may work with some of the embodiments for analyzing the relationship between diaper wear with sleep and/or developmental indicators as illustrated in FIG. 2. The results obtained by the embodiments of computer system 100 which may be used in embodiments of any of: verification module 206, sleep initiation monitoring module 208, or sleep evaluation module 210, may be displayed via display 212 or any other display, such as a printout, or any other output form which is visible to a user.

Referring to FIG. 2, an analysis system 200 may include any modules of computer system 100 in any of the following specific purpose machines described below including the verification module 206, sleep initiation monitoring module 208, and sleep evaluation module 210. FIG. 2 illustrates an embodiment of an analysis system 200. As illustrated in FIG. 2 the analysis system 200 may include a verification module 206, a sleep initiation monitoring module, and a sleep evaluation module 210. Other modules capable of functionality described below may also be included in the analysis system 200.

For example, a diaper securing device may include a device that would secure or assist in securing a diaper to a subject. Alternatively, the securing of the diaper may be implemented using a human operator or a combination of an assistance device with the human operator. Further, the subject to which the diaper is secured may be human or other species.

For example, a diaper securing assistance device may be a diaper verification device, such as an interactive display or a user interface form that is used to accept verification data that indicates that the subject is secured with a specified diaper. A diaper securing verification device may be implemented with any part of computer system 100 and a verification module 206. Generally, the term “module” is used to include any combination of hardware and/or software. Thus, the verification module 206 may be software alone that is stored in any of the memory of system 100. Alternatively, the verification module 206 may be a separate hardware device all together. Those with ordinary skill in the art of hardware and/or software will understand the different embodiments of a module necessary to embody the specific purpose verification module 206.

The verification module 206 is a specific purpose module that takes as input an indication that the proper diaper has been successfully secured to a subject. Such a diaper includes a cloth diaper or a disposable diaper. The verification module 206 is a specific purpose module that causes this information to be stored or transferred for further processing as required by the particular embodiment of the analysis system 200. For example, the diaper wear verification module 206 may obtain information from a user. For example, the diaper wear verification module 206 may be a user interface or a specific user interface form used to collect verification information. The information may indicate that the appropriate test diaper was secured to the test subject. This information may be transferred to a sleep initiation monitoring module 208. For example, after sleep initiation monitoring 208 module 208 receives the verification input, the operation of the sleep initiation monitoring module 208 may begin. For example, after the sleep initiation monitoring module receives information that sleep has been initiated by the subject, information may be sent to a sleep evaluation module 210 so that the sleep evaluation module may begin collecting data of the sleep of the subject. After the sleep data is collected, the sleep evaluation module 210 may have the sleep data evaluated or displayed for evaluation.

A sleep initiation monitoring module 208 may be a part of the analysis system 200. Generally, a sleep initiation monitoring module 208 may be software alone that is stored in any of the memory of system 100 or a separate hardware device all together or anything in between. Those with ordinary skill in the art of hardware and/or software will understand the different embodiments of a module necessary to embody the sleep initiation monitoring module 208 functionality. The sleep initiation monitoring module 208 may fall into several types.

The first type of sleep initiation monitoring module 208 may include a laboratory sleep environment where sleep is monitored by a lab attendant and/or a sleep monitoring device. The second type can record similar data as the first type of sleep monitoring devices, however, the second type of monitoring devices can be used outside the laboratory and may not need an attendant. For example, the second type of sleep initiation monitoring module 208 may be found in a subject's home. Further, the second type of sleep initiation monitoring module 208 may be portable. The third type of sleep initiation monitoring module 208 may transfer the data immediately without recording the sleep data.

For example, a simpler embodiment of a sleep initiation monitoring module 208 may include only a user input module, wherein the user uses his observation techniques to determine when the subject has fallen asleep and then the user provides sleep initiation input to a sleep initiation monitoring module 208.

Another more complicated embodiment of a sleep initiation monitoring module 208 includes a video analysis indication that the subject has fallen asleep. In particular, a video monitoring device, such as 128, along with detection software may be used to detect the closing of the eyes of the test subject.

Anther example of a sleep initiation monitoring module 208 includes a polysomnography (PSG) module. A polysomnography module records the biophysiological changes that occur during sleep. The recordings are usually performed at night, when most subjects sleep, though some labs can accommodate testing at other times of day. The PSG modules may monitor many body functions. The body functions that can be monitored include brain functions, such as global neural encephalographic activity, by using an electroencephalogram (EEG) from electrodes placed on the patient's scalp, eye movements by using an electrooculogram (EOG) from electrodes placed near the outer canthus of each eye, muscle activity or skeletal muscle activation by using an electromyogram (EMG) from electrodes placed over the mentalis, submentalis muscle, and/or masseter regions, heart rhythm by using an electrocardiogram (ECG) with chest leads, respiratory effort, by chest-wall and abdominal movement via strain gauges, piezoelectric belts, inductive plethysmography, impedance or inductance pneumography, endoesophageal pressure, or by intercostal EMG, nasal and/or oral airflow via thermistor or pneumotachograph, oxygen saturation (Sp02) via pulse oximetry, body position via mercury switches or by direct observation, limb movements (arms and legs) via EMG, recordings of or vibration (frequency and/or volume) may be recorded, end-tidal C02, transcutaneous C02, esophageal pH, penile tumescence, and bipolar EEG.

A polysomnogram module may have eleven channels requiring a minimum of 22 wire attachments to the patient. Two to four channels may be for an electroencephalogram (EEG), one or two may measure airflow, one may be for chin movements, one or more for leg movements, two may be for eye movements (EOG), one may be for heart rate and rhythm, one may be for oxygen saturation and one may be for a belt which measures chest wall movement and one may be for a belt which measures upper abdominal wall movement. Wires for each channel of data lead from the patient and converge into a central box, which in turn is connected to a computer system, such as system 100, for recording, storing, and displaying the data. During sleep the computer monitor may display multiple channel data continuously. In addition, some labs may have a small video camera in the room so the technician may observe the patient visually from another room.

Alternatively, the sleep initiation monitoring module 208 may include an actigraphy module. Actigraphy modules may be better suited for cases where longitudinal or large scale data sets need to be generated, or when a PSG module is not a cost-efficient option. Actigraphy modules may be used in conjunction with the polysomnography modules or instead of the polysomnography module as part of the sleep initiation monitoring module 208.

Actigraphy modules inlcude an actimetry sensor. An actigraph module may inlcude a piezoelectric accelerometer to measure non-gravitational accelerations, a low pass filter which may be used to filter out noisy vibrations, a timer to start/stop the module at specific times, a memory, and an interface, such as a serial bus interface, etc.

An actigraphy module may have a number of different methods of accumulating the values from the accelerometer in memory including a zero crossing mode (ZCM) module which counts the number of times the accelerometer waveform crosses 0 for each time period, or a proportional integral mode (PIM) module which measures the area under the curve, and adds that size for each time period, or a time above threshold (TAT) module which uses a certain threshold, and measures the length of time that the wave is above a certain threshold. The actigraphy module may be any one of a zero crossing computing device, a proportional integral computing device, an area under the curve computing device, a time above threshold computing device, and a length of time that the wave is above a certain threshold computing device.

The actigraphy module includes embodiments of any of the actgraphy devices manufactured by the following: ActiGraph LLC, ActiTrainer (was CSA/MTI), Ambulatory Monitoring Inc., Motionlogger Actigraph, Cambridge Neurotechnology Actiwatch, Consultoria Eletronica Four Parameter Brazilian Actigraph, Gulf Coast Data Concepts USB Accelerometer, MetriSense, Inc.—Distributor of activity monitors from CamNtech, Ltd., UK., PAL Technologies Ltd—Manufacturer of activity monitors, Philips Respironics Actiwatch.

The sleep initiation monitoring module 208 may be any one or more of a group consisting of: a polysomnography device, an actigraphy device, or a video monitoring device, or a remote sensing device that utilizes Doppler radiofrequency for remote sensing of physical activity and respiratory rate. Also, both the actigraph and/or the remote sensing device may be coupled to software and/or computer-based computational algorithms and may be specifically developed for the remote sensing device or actigraph. The software and/or computer-based computational algorithms may enable processing of the physical motion signals captured by the sleep initiation monitoring module 208, such as an actigraph and/or the physical motion and/or respiratory signals captured by the sleep initiation monitoring module 208, such as a remote sensing device to determine the sleep/wake status of the individual under examination. For example, both the software and/or computer-based computational algorithms, such as the software for the actigraph and the algorithm for the remote sensing device, may enable user-dictated modification or modulation of the algorithm parameters that affect the determination of sleep/wake status based on the activity and/or respiratory rate data.

The sleep evaluation module 210 may usurp all the functions of the sleep initiation monitoring module 208 or may include many of the same sub-modules as the sleep initiation monitoring module 208. For example, some of the same input modules may be used to gather data for evaluating sleep as the data that was gathered to determine if and when sleep initiation ocurred by the subject. The sleep evaluation module functions of the sleep evaluation module 210 may be initiated after it receives a signal from the sleep initaition monitoring module 208. The data gathered and the data displayed by the sleep evaluation module 210 may be different from the data gathered and displayed by the sleep initaition monitoring module 208. For example, sleep initaition data may only inlcude breathing data, whereas sleep evalution data may inlcude different, such as movement data and/or heart rate data.

Other embodiments of a sleep evaluation module 210 inlcude a survey questionnaire user interface or a diary user interface or both. For example, a sleep survey questionnaire may be filled out by a user and that data may be entered via a sleep evaluation module 210 that takes the input and translates it to stored data and/or captures the survey data directly on to an electronic data capture device, such as a modified palm pilot or personal digital assistant or any other user interface software and/or hardware. Other embodiments of a sleep evaluation module 210 may inlcude a user interface specifically directed to ask the user to enter sleep evlaution data, such as a form with input receivers.

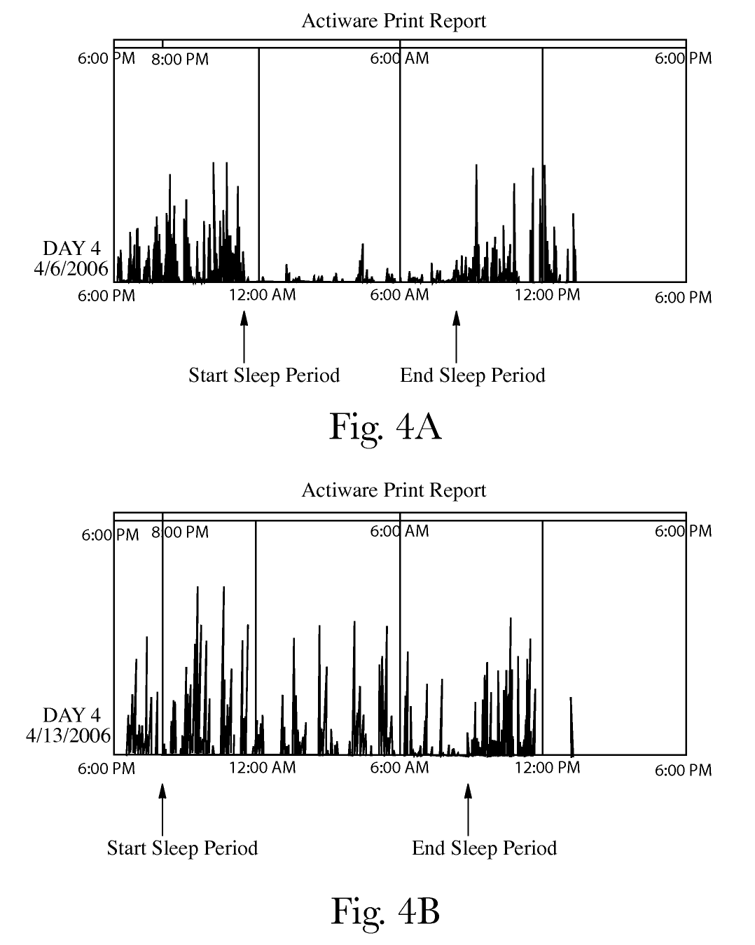

The sleep evaluation data that may be gathered by the sleep evaluation module 210, and may be interpreted and processed to provide more easily understood data, such as the data presentation described below in FIGS. 4A and 4B.

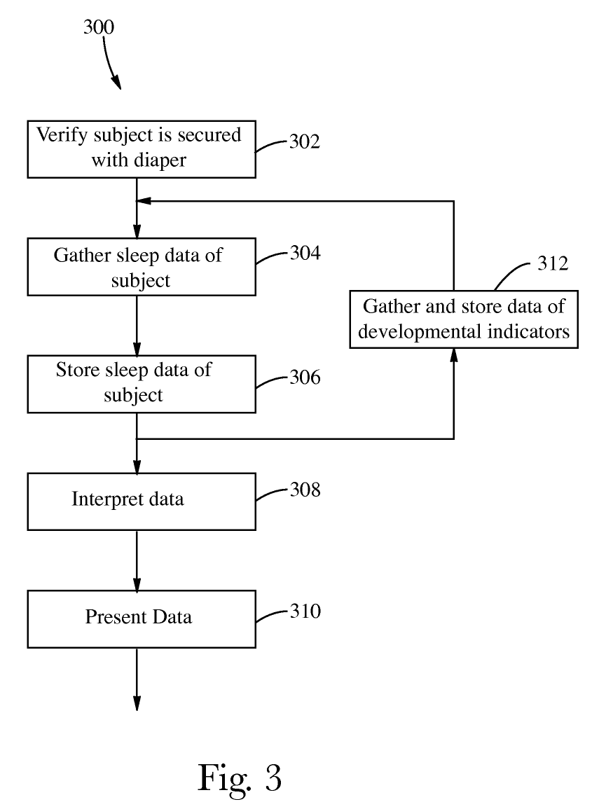

FIG. 3 illustrates an embodiment of a process 300 for facilitating analysis of the relationship between diaper wear with sleep and/or developmental indicators. The process 300 includes verifying that the subject has been secured with a diaper (block 302) as well as monitoring for an indication that the subject has fallen asleep (block 304). The process also includes storing data that may be received from a sleep evaluation module 210, such as an actigraph sensor (block 306). In some embodiments, the actions performed in blocks 304 and 306 (as shown in FIG. 3) may be performed by the sleep evaluation module 210 alone or with the conjoined action of a user entering data into the sleep evaluation module. Alternatively, the action in block 304 may be performed by a sleep initiation monitoring module 208 and the action in block 306 may be performed by a sleep evaluation monitoring module 210. Blocks 304 and 306 may be substituted entirely for block 312 so that diaper wear and developmental indictor data can be evaluated without doing any sleep evaluation if the association between diaper wear and sleep may be assumed. Interpreting the stored data is optional (block 308). The action performed in block 310 is optional and includes presenting the data in a more easily understandable presentation such as is illustrated in FIGS. 4A and 4B. The action performed in block 310 includes the presentation of the data as illustrated below in Tables 1-13. The tables are embodiments of a user interface presentation module that may be included in analysis system 200.

Optionally, the process may in addition include gathering and storing data related to developmental indicators of the subject (block 312). This data may be gathered and stored during the sleep evaluation data gathering and storing cycle. The action shown in block 312 may be performed by a developmental indicator test module which is a specific purpose computing module that is used to gather and store user input of developmental indicator data, such as developmental indicator data that is entered into an input interface.

Other embodiments of the machine, manufacture, and process are too numerous to list. Other embodiments are disclosed in the following references, which are incorporated herein by reference, in their entirety: “Comparative evaluation of sleep quality and cognitive behaviors among Chinese infants, 2 to 9 month old, with use of traditional cloth diapers or PAMPERS® disposable diapers disposable diapers.” by Odio et al., “Sleep and Cognition in Chinese Infants” by Bauer at al., “A Good Night's Rest Provides More than Beauty Sleep: The Effect of Nighttime Sleep Habits on Caregiver Ratings of Infant Temperament in China” by Lukowski et al., and “A clinical investigation of sleep and cognition in Chinese infants, as a function of nighttime sleep patterns” by Bauer et al. These publications include embodiments of the analysis system 200.

Presented below are some embodiments from the above listed references. For example, FIG. 4A and FIG. 4B illustrate embodiments of a user interface of sleep evaluation module 210 used in block 310 to present the gathered sleep data. Other embodiments of analysis system 200 may be used to implement the example analysis systems below. Example analysis systems 1, 2, 3 are listed together as their data is similar. Example analysis system 4 is an investigation of performance in specific developmental indicators such as indicators in cognitive, linguistic, and social domains as a function of sleep practices. Example analysis system 5 investigates the effect of diapers on infant sleep habits and on the development of temperament.

Example Analysis Systems 1, 2, 3 include end-point measurement data which was accomplished as follows:

1. Sleep Evaluation Data as Sleep Quality Data

Sleep quality was evaluated in several ways. First, study personnel visited the families of infant participants in both groups thirteen times over the course of the six-week diaper manipulation. At those visits, caregivers completed Home Visit Questionnaires, in which they were asked whether they placed their infant in their assigned type of diaper the previous night and how many times the infant was disrupted the previous night for diaper changes or other reasons. Second, mothers completed detailed Nighttime Sleep Diaries for three consecutive nights during either the fourth or fifth week of the diaper manipulation to indicate when the infant was put to bed at night and when the infant woke the next morning; the number of times the infant was disrupted during the night, and whether the infant awoke or not as a result of the disruption. Mothers were provided with standard, luminous clocks and asked to record each event immediately after its occurrence, thereby providing for the most accurate self-report measure possible. Third, in a subset of 20 children in the cloth diaper and another 20 children in the PAMPERS® or disposable diapers group actigraphy measures were taken using actigraphs in parallel with the application of the detailed sleep inventory. Actigraphy recordings were taken, analyzed and interpreted, following the same procedures as described for Study 2. The data taken by the Home Visit Questionnaires, Nighttime Sleep Diaries, and the Actigraphy recordings are sleep evaluation data.

2. Developmental Indicator Data—as Child-Mother Interaction Data

The relationship that an infant has with her or his mother is critical not only for the physical safety and security of the infant but for his or her psychological well being and later peer acceptance. Mother-infant dyads differ in the amount of synchrony they display during their face-to-face interactions. By noting the details of the facial behavior (looks to partner, looks away) and vocal behavior (baby vocalizes, mother calls baby's name) produced by mother and baby, and especially the interplay between these different behaviors, it is possible to determine the level of behavioral synchrony within the dyad. These measures are an indication of early quality of nonverbal communication, which is in turn likely to affect infants' emotion regulation and emotional development more generally. Thus, in the present study, the infant-mother interaction was recorded using videocameras 128 and the video was coded for visual and vocal behavior. Dependent measures included the number of bouts of shared attention, maternal strategies for engaging infants, the number of looks to partner and looks away, and the number vocalizations by mother and baby.

The quality of the child-mother interaction was evaluated twice, once at the beginning of the analysis, before the children were assigned to specific diaper conditions and again at the end of the 6-week period of diaper use. Both evaluations were done in identical, free-interaction settings in which the mother was asked to engage in play with her child, as she would during a normal day at home. To stimulate the interaction, the mother was given, at the beginning of each interaction session, 3 age-appropriate toys: a rattle, an octopus and a caterpillar and was also given the following instructions: “We would like you to play with your baby using these toys as you would if you had a few minutes of extra time at home. Please try to play with one toy at a time. We would like to see how you and your baby normally play together with these kinds of toys. We'll let you know when to stop playing.” The order, sequence or how each mother and child played with the toys was not directed in any way. Each mother and child were allowed to play freely and unrestricted, with the toys provided, for a total of four minutes.

Each interaction session was recorded, in its entirety, using two independent video cameras 128. One camera was focused on the child at all times; the other on the mother. A picture-in-picture set-up was used such that the screen that captured the mother's face appeared within the main screen which was directed at the child. This resulted in a video situation that displayed, simultaneously, on the same screen, both the child and the mother, their full facial expressions, direction of attention and other important attributes of the interaction established between them during the four-minute play session. The video record of each session was transported to the Cognitive Development Laboratory facilities at Duke University in the USA. Each video was coded by trained laboratory personnel who had had no prior contact with the participants or other key aspects of the execution of the study and who, most importantly, were blinded as to the diaper assignment of each child.

The coding of each interaction session was anchored on the baby's state of attention, that is to say, on where and how the child's attention was directed (for example, attention directed to the toy, to the mother, to other elements, or attention was inactive or the emotional state of the child negative, i.e., crying, disgust, etc.). The mother's behavior was analyzed subordinate to the child's attention, i.e., mother maintaining the child's attention, redirecting his/her attention, etc.

More specifically, each mother-child interaction session was coded in three steps: (a) Child's state of attention; (b) Source of change in child's state of attention, and; (c) mother's response. Examples of this coding scheme or developmental indicator data are provided in Table 1. The coding of the Child-Mother interaction is developmental indicator data.

TABLE 1
Examples of the Coding Scheme for Child - Mother Interaction Exercise

| Steps | Focus |
|---|---|
| A. Child's state of | Toy - active manipulation; looking only; inattention |
| attention | Mother - looks at mother only; looks at her while |
| | holding object |
| | Other - attention not on mother or object |
| | Negative affect - crying; fuzzing; rejects object/ |
| | mother's attention |
| B. Source of | Mother - change due to mother's actions |
| change in child's | Child - change initiated without moter's intervention |
| attention | Other - change due to environmental factor, ie., |
| | noise, disturbance, etc. |
| C. Mother's | Maintain - maintains child current attention focus |
| response | Redirect successful - mother's successfully changes |
| | child's attention focus |
| | Redirect unsuccessful - mother tries but does not |
| | change child's attention |

3. Developmental Indicator Data as Infant Temperament Data

Infants differ with respect to their behaviors and reactions in everyday and novel situations. Some of the ways in which they differ have implications for social and cognitive development. For example, infants who are more sociable find it easier to approach novel people and objects and interact with them. These infants tend to perform at higher levels on memory tasks. Infants also vary in their ability to regulate their own behavioral states and emotional reactions. Better behavioral self regulation is associated with later moral development as well as academic achievement. As in the case of language and gesture development, because parents see their infants in many more contexts and over much longer periods of time than researchers can, valid and reliable parent report measures of temperament have been developed.

In this analysis, infant temperament was evaluated using the Infant Behavior Questionnaire-Revised (IBQ-R) data by Gartstein & Rothbart. Caregivers completed translated versions of the IBQ-R data both before the initiation of and after completing the six-week diaper manipulation. The IBQ-R data is a 191-question parent-report instrument that includes fourteen dimensions of temperament or developmental indicator data (shown in Table 2) based on the definition reported in Rothbart and Derryberry. As described in Gartstein and Rothbart and in Gartstein, Slobodskaya, and Kinscht, the construction of the inventory of data was based on operational definitions of the various temperament dimensions as well as on item-specific and conceptual analyses to ensure internal validity and minimize conceptual overlap among the scales. Caregivers of infant participants completed the IBQ-R data as instructed, circling their responses on a 7-point scale to indicate the frequency with which their infants performed in specific manners over the seven days prior to receipt of the questionnaire. The temperament data obtained from the IBQ-R was reduced into the fourteen scales shown in Table 2 according to the data reduction instructions obtained with the inventory. Scales were computed separately for the first and second administrations of the questionnaire interface.

TABLE 2
Names and Descriptions of Scales of the IBQ-R data

| Temperament Scale | Description |
|---|---|
| Activity level | Gross motor activity |
| Distress to limitations | Fussing, crying or experience of distress in caretaking activities, |
| | when confined, or in other situations |
| Fear | Startle or distress reactions when presented with changes in stimuli; |
| | inhibited approach to novelty |
| Duration of orienting | Ability to orient to single stimuli for extended durations of time |
| Smiling and laughter | Smiling and laughter exhibited in everyday or caretaking activities |
| High pleasure | Amount of pleasure received from high-intensity stimulation |
| Low pleasure | Amount of pleasure received from low-intensity stimulation |
| Soothability | Ability to be soothed upon comforting by the caregiver |
| Recovery from distress | Ability to recover from peak excitement or distress; |
| | ease with which infant fell asleep |
| Cuddliness | Enjoyment of caregiver contact; molding to the body of the |
| | caregiver |
| Perceptual sensitivity | Ability to notice slight, low-intensity environmental stimuli |
| Sadness | Lowered mood relating to suffering, loss, |
| | inability to perform in desired ways |
| Approach | Excitement and positive anticipation of pleasurable activities |
| Vocal reactivity | Amount of vocalization exhibited in daily activities |

Example Analysis System 1

Sleep Evaluation of Subjects Secured with Cloth Diapers

A study was conducted to determine the frequency of nocturnal sleep interruptions as evaluated by a sleep evaluation module 210. The sleep evaluation data included data of sleep interruptions experienced by the children in the study, resulting from the need to change their cloth diapers. Enrolled in the study were infants 3 to 9 months old, in general good health, whose parents indicated that the child had used cloth diapers exclusively for at least the 2 weeks prior to study enrollment. A total of 694 children, recruited from 5 different cities (approximately 140 children in each city) completed the study. For study execution, a team of trained nurses lived in each of the study cities (Xianyang, Deyang, Huaian, Xiangtan and Xingtai) for a 1-week period. Each child in the study was visited twice in his/her home. The first visit to screen the child for acceptance into the study; the second visit by a nurse who asked the mother of the child to answer a series of questions that described the number and reason for sleep interruptions of her child during the previous night. The answers to the questions are an embodiment of the user input which a sleep evaluation module accepts via a user interface. In this way a sample of sleep evaluation data was obtained from a large sample of children in the five cities included in the study.

A total of 694 children, three to nine months of age completed the study. Of this total, 359 (52%) were male and 335 (48%) were female; 323 (47%) were between three-six months of age and 371 (53%) ages between six and nine months. Similar male/female and age ratios were obtained among the five cities in which the study was executed.

The results of the sleep analysis system 200, which summarize the answers of the mothers in the study to the question—“How many times did you change your baby's diaper last night?” are presented in Table 3. Among the total sample interviewed, more than 50% of the mothers reported changing their child's diaper three or more time per night and a sizable proportion of nearly one third of the mothers reported four or more changes per night. Conversely, only 15% of the mothers reported one or two diaper changes per night. Additionally, about 90% of the mothers interviewed reported that their child “usually” wakes up when she does a diaper change during the night. Therefore, the results of this survey input information indicate that frequent disruptions of the child's nighttime sleep are commonly associated with the traditional Chinese practice of using cloth diapers.

TABLE 3
Frequency of nighttime diaper changes. The data are shown
as the percent of all the mothers interviewed
who reported the specific number or diaper
changes given in the X axis.

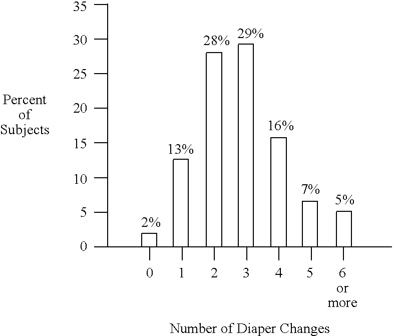

Example Analysis System 2

Comparative Analysis of Sleep Evaluation Between Subjects in Cloth Diapers Versus Subjects in Disposable Diapers

A randomized, clinical intervention analysis which includes a comparison of the effects on sleep quality of the use of traditional Chinese cloth diapers or PAMPERS® or disposable diapers Super Dry disposable diapers, a commonly sold infant diaper in China was run. The analysis was conducted in two cities, Liaoyang and Jinzhou and took a total of 7-weeks to complete, in each city. The first week was devoted to subject screening and study preparations. The following 6 weeks constituted the sleep comparison based on diaper phase of the study. Eligible for enrollment were infants 2 to 7 months old, in general good health, whose parents indicated that the child had used exclusively cloth diapers for at least the 2 weeks prior to study enrollment. Following enrollment into the study, each child was randomly assigned to continue using his/her usual cloth diapers or switch to exclusive use of PAMPERS® or disposable diapers, which were provided to the child free of charge, for the 6 weeks duration of the product use period of the study. Parents agreed to exclusive use of their assigned diaper for the duration of the study. A team of trained nurses resided for the period of study duration, first in Liaoyang and then in Jinzhou to execute the study. In each city approximately 150 children were enrolled in the study.

The nurse assigned to each child visited his/her home two times per week to enter sleep evaluation data. At each visit, the nurse asked the mother a series of questions that described the number and reason for sleep interruptions of her child during the previous night. This provided a longitudinal record of nocturnal sleep interruptions, consisting of 12 sequential applications of the questionnaire for each child over the 6 week product use phase of the study.

Beginning on the third week of the study, a subset of 120 children were randomly selected from the total study sample (50% cloth; 50% PAMPERS® or disposable diapers users) to obtain an objective, instrumental record of their sleep profiles. To do this, an actigraph device was applied to the child's right ankle and kept in place for 3 consecutive nights. The actigraph is a sensor that responds to changes in acceleration and thus, detects the pattern of motion of the child during the night. It is well recognized that the most sensitive and reliable method to evaluate sleep is by polysomnography (PSG). It is also well understood that PSG is a complex, expensive and intrusive methodology that is difficult to use in young children and which can only be applied in the laboratory, not in the home. On the other hand, an extensive literature in the areas of sleep medicine and sleep research validates that actigraphy can provide a reliable and acceptable approximation to PSG. Thus, actigraphy based measurements provide a reasonable indication of the sleep quality of the child and, in the case of at-home studies such as the present one, offer the only viable objective measurement approach.

The graphs in FIGS. 4A and 4B, illustrate examples of two child-nights of actigraphy recordings, the left graph depicting a night with prevalent uninterrupted sleep (FIG. 4A), the right one a night in which the child experienced multiple arousals and disruptions of sleep (FIG. 4B).

The actigraphy data from each child was downloaded from the actigraph into electronic files that were transferred in blinded manner to the Laboratory of Sleep and Functional Neurobiology of the University of Chile. The first night of actigraph recordings for each child was discarded as an “adaptation night” and only the records from nights two and three from each child were used in the analysis of the results. The actigraphy signals were decoded using appropriate algorithms for the conversion of motion data into sleep quality parameters, specifically: number of sleep episodes/night, duration of sleep episodes, number of wake episodes/night, duration of wake episodes, total sleep/night and total time awake/night.

In this clinical analysis, the sleep evaluation module 210 obtained actigraphic recordings of the sleep quality of children who routinely used cloth diapers or children who changed to use of PAMPERS® or disposable diapers. The actigraph recordings were obtained on weeks three and five of the analysis and the results were pooled for all cloth or PAMPERS® users. Out of the 120 children who participated in the analysis, 54 in the PAMPERS® group and 58 in the cloth group had actigraphic recordings that could be reliably evaluated. The actigraphy results are summarized in Table 4 which is an embodiment of a user interface of sleep evaluation module 210.

As noted in the Table, there were no differences in terms of the beginning or end of the nocturnal sleep period between the two groups of children. Children assigned to the PAMPERS® group had significantly superior outcomes in several important sleep quality parameters compared to children in the cloth diaper group. The sleep percentage (sleep efficiency) was higher and the wake percentage lower for children using PAMPERS® than for those using cloth. Importantly, the average duration per sleep episode was substantially longer among PAMPERS® than cloth users and the number of wake episodes was reduced in children using PAMPERS®. On average, as well, the longest sleep episode for each child during the night was significantly higher for PAMPERS® users than for cloth users. In summary, these results indicate that use of PAMPERS® was associated with significantly improved nocturnal sleep outcomes compared to use of cloth diapers.

TABLE 4
Quality of Nocturnal Sleep Measured by Actigraphy Among Children
2-7 Months of Age Users of Traditional Cloth or PAMPERS ®
Disposable Diapers. (The data are presented as the Mean ± Standard
Deviation of the number of children in each treatment group. Statistical
comparisons are by Student's t test.)

| | PAMPERS | CLOTH | P |
|---|---|---|---|
| | (n = 54) | (n = 58) | value |
| NOCTURNAL PERIOD | 20:41 ± 1:16 | 20:50 ± 1:19 | NS |
| ONSET TIME (hh:mm) | | | |
| NOCTURNAL PERIOD END | 6:56 ± 1:02 | 6:58 ± 1:00 | NS |
| TIME (hh:mm) | | | |
| SLEEP PERCENTAGE | 86.4 ± 7.3 | 83.4 ± 7.1 | <0.02 |
| WAKE | 13.6 ± 7.3 | 16.5 ± 7.1 | <0.02 |
| PERCENTAGE | | | |
| SLEEP EPISODES NUMBER | 6.2 ± 2.1 | 6.9 ± 2.0 | NS |
| WAKE EPISODES NUMBER | 5.3 ± 2.1 | 5.9 ± 2.0 | <0.04 |
| MEAN SLEEP EPISODES | 100.6 ± 66.6 | 82.5 ± 38.6 | <0.01 |
| DURATION (min) | | | |
| MEAN WAKE EPISODES | 16.7 ± 11.8 | 17.4 ± 7.0 | NS |
| DURATION (min) | | | |
| LONGEST SLEEP EPISODE | 240.4 ± 114.8 | 187.6 ± 79.7 | <0.01 |
| (min) | | | |
| LONGEST WAKE EPISODE | 68.0 ± 79.3 | 70.5 ± 79.0 | NS |
| (min) | | | |

Example Analysis System 3

Comparative analysis of sleep evaluation and developmental indicators, such as cognitive behaviors among Chinese children using cloth or PAMPERS® or disposable diapers.

Analysis system 200 may gather data for the following reasons: (a) to confirm findings from Study 2 on the benefits of PAMPERS® or disposable diapers use to improve infant sleep quality, and: (b) as an initial evaluation of the hypothesis that children who experience fewer interruptions of nocturnal sleep may perform better in specific interactive behaviors that are important in a developmental indicator, such as cognitive development of children. Specifically, the primary end-points evaluated in the analysis may be: (a) actigraphy measurements and sleep survey questionnaire inputs to determine the sleep quality profiles of the children; (b) child-mother interaction test data to evaluate how the child relates to and learns from his/her mother, and; (c) an Infant Behavior Questionnaire data that measures infant temperament (how the child relates to his/her environment).

The analysis was conducted as a six-week, randomized, clinical comparison between two treatment groups. Eligible for enrollment in the study as subjects were infants who were 5.5±0.5 months of age on the first day of the study and who met the following additional enrollment criteria: were in general good health; had been exclusive users of cloth diapers for at least the past 7 days; the child's parent declared that the child routinely had no less than 4 diaper changes per night; and the parent of the child agreed to exclusive use of their assigned diaper for the 6 week duration of the study.

A total of 82 children who met the above criteria were enrolled and completed the 6 week study, which was executed in the cities of Jinzhou (n=43 infants; 20 girls) and Shenyang (n=39 infants; 19 girls) in Liaoning province. On enrollment into the analysis, each child was randomly assigned to one of two possible diaper use groups: one half of the children were assigned to continue use of their traditional cloth diapers; the other half were assigned to use PAMPERS® or disposable diapers BABY DRY® diapers (note, in China, the Chinese characters (kanji) are registered) which is a commercial product that has been sold in China for several years. Parents assigned to the cloth diaper group were instructed to continue with the normal diapering practices and verification indication that they had been following to that point. Parents of infants assigned to the PAMPERS® or disposable diapers group received from the analysis personnel, a demonstration and information about the diapers' absorbency, effectiveness in keeping wetness away from the skin and maintaining skin health. This enabled the parents to understand that changing the disposable diaper less frequently (or not at all) overnight would not jeopardize their baby's skin health. It was also made clear to the parents that it would be their decision entirely how often to change their baby's diaper in the night.

Once the analysis initiated, each subject's family was visited by analysis personnel once per week. During this visit, analysis personnel interviewed the child's mother to complete a Home Visit Questionnaire that included questions about treatment compliance and a survey of the child's previous night's sleep interruptions. This data may be input to sleep evaluation module 210. At this visit also, subjects in the PAMPERS® or disposable diapers group received, a new supply of diapers for their use, as needed.

In this analysis, sleep quality of children using cloth or PAMPERS® diapers was evaluated to examine the relationship between improved sleep and performance in child-mother and temperament assessments. Sleep quality was evaluated both by survey questionnaire and by actigraphy. The results from the analysis provide convincing evidence of superior sleep quality with PAMPERS® diapers versus cloth diapers and of the positive impact of better sleep on the cognitive performance end points examined. The specific results are presented in the following 3 sections.

1. Sleep Quality Results:

The results in Table 5 present the actigraphy outcomes for children using PAMPERS® or cloth diapers. As noted in the Table, children using PAMPERS® had longer duration of sleep episodes, less overall time awake and shorter wake episodes than those using cloth diapers. In addition there was a strong trend for longer overall sleep time among PAMPERS® compared to cloth users.

TABLE 5
Quality of Nocturnal Sleep Measured by Actigraphy Among Children
Using Traditional Cloth or PAMPERS ® Disposable Diapers.
(The data presented are the Mean ± Standard Error of the number
of children in each treatment group. Statistical comparisons are by
Student's t test).

| | Cloth diapers | PAMPERS ® | |
|---|---|---|---|
| Measure (minutes) | (n = 19) | (n = 19) | p value |
| Overall sleep time | 495.9 ± 9.5 | 527.5 ± 16.8 | 0.10 |
| Mean duration of sleep | 99.5 ± 10.7 | 157.9 ± 32.9 | 0.05 |
| episodes | | | |
| Overall time awake | 99.0 ± 10.5 | 61.5 ± 7.4 | 0.05 |
| Duration of waking episodes | 20.3 ± 1.7 | 14.8 ± 1.6 | 0.05 |

Table 6 summarizes the sleep quality results for children analyzed, as reported by mothers in their sleep diaries during the same 3 nights as the actigraphy measures were taken. Sleep surveys were completed by the full sample of study participants, including the subset that participated in the actigraphy measurements shown in Table 4. As noted in the table, children using cloth diapers were reported by their mothers to have had significantly more waking episodes, more total disruption and to have spent more total time awake during the night compared to those using PAMPERS® disposable diapers.

TABLE 6
Maternal Diary Reports on Sleep Quality Among Children
Using Traditional cloth or PAMPERS ® Disposable
Diapers. (The data presented are the Mean ± Standard Error of the
number of children in each treatment group).

| | Cloth | PAMPERS ® | |
|---|---|---|---|
| Sleep Measure | Diaper (n = 41) | (n = 41) | P value |
| Number of waking episodes | 3.69 ± .18 | 1.66 ± .18 | 0.05 |
| Number of total disruptions | 4.99 ± .14 | 2.08 ± .14 | 0.05 |
| Time spent awake | 82.58 ± 6.64 | 35.90 ± 6.64 | 0.05 |

In summary, whether measured by actigraphy or parental survey report, use of PAMPERS® diapers was associated with significantly improved sleep quality compared to use of traditional cloth diapers.

2. Mother-Child Interaction

The results in Table 7 present the child-mother interaction or developmental indicator test outcomes. The strongest finding from this evaluation was a positive association between improved sleep quality and better child-mother interactions. The positive associations were specific for comparisons between sleep quality and interactive outcomes, not for differences between treatment groups. That is to say, due to sample size and variability in the experiment, a direct statistical difference between outcomes in the PAMPERS® vs. cloth groups was not found. However, the sleep quality result data as may be displayed on a user interface is shown in Tables 5 and 6 show that children in the PAMPERS® group had demonstrably superior sleep profiles. Therefore, the positive associations between sleep and interactive outcomes are primarily due to the results of the children in the PAMPERS® group, not those in the cloth group. In other words, better sleep results in better child-mother interactions; PAMPERS® delivers the quality sleep to achieve these superior interactions in a higher proportion of children compared to the use of cloth diapers.

TABLE 7
Positive Associations Between Sleep Quality Parameters and
Improved Mother Child Interactions. (The results show statistically
significant correlations between sleep quality and interactive
outcomes).

| Child-Mother | | |
|---|---|---|
| Interaction Parameter | Sleep Quality Measure | p value |
| Longer duration of toy | Fewer nocturnal sleep disruptions | 0.04 |
| interaction with mother | | |
| Longer active episodes with | Fewer nocturnal sleep disruptions | 0.07 |
| mother | | |
| Mother maintains child | Longer durations of sleep episodes | 0.04 |
| attention better | Fewer nocturnal sleep disruption | 0.03 |
| Less time baby in negative | Longer durations of sleep episodes | 0.04 |
| affect | Fewer nocturnal sleep disruptions | 0.05 |
| Shorter duration of negative | Longer durations of sleep episodes | 0.04 |
| affect episodes | | |

3. Infant Temperament

The infant temperament results were analyzed by the computation of correlations between measures obtained from the Nighttime Sleep Diaries and from the second administration of the IBQ-R because this approach allowed, most effectively, the establishment of a relation between infant sleep habits and infant temperament.

It was found in this study that infants who experienced a greater number of nighttime disruptions or who were disrupted for greater amounts of time exhibited higher rates of gross motor activity, were more distressed when placed in limiting situations, and more eagerly and readily anticipated positive activities. Additionally, infants experiencing these higher frequencies and/or durations of nocturnal disruptions also were less likely to fall asleep easily, to quickly recover from peak excitement and to perceive low-intensity environmental stimulation. Conversely, children with superior sleep profiles also had superior temperament outcomes in each of the above scales.

In addition, the temperament results indicated that changes in sleep routine of the infants associated with use of cloth or PAMPERS® affected the development of specific temperament variables over time as determined by comparisons between the first and second administrations of the IBQ-R. For example, infants in the cloth diaper control group received significantly higher ratings over time on activity level, distress to limitations, and approach, whereas for infants in the PAMPERS® group significant increases over time were found on the scales of activity level, perceptual sensitivity, and vocal reactivity.

While the development over time results require further investigation to be properly interpreted in terms of their significance and possible implications, the temperament data do suggest, consistent with the mother child interaction data, that better sleep, overall, associates with behavior in social settings that is more conducive to positive interactions in learning situations.

Example Analysis System 4

Example analysis system 4 is an investigation of performance in specific developmental indicators such as indicators in cognitive, linguistic, and social domains as a function of sleep practices among infants 6 to 7½ months of age. It involves a treatment (use of disposable diapers) that is expected to encourage achievement of more regular sleep patterns among infants and thus to be associated with higher levels of performance on the target tasks. The question of whether more regular sleep patterns among infants are associated with higher performance on specific tasks was addressed by comparing scores on the tasks by groups of infants in the treatment and no-treatment control groups. The groups were not expected to differ on the tasks at the beginning of the treatment. By the end of the 6-week treatment, differences were expected with higher performance by infants in the treatment group. The study also featured a test of whether sleep facilitates consolidation of new learning in infants. This question was addressed by administering the study phase of three tasks on Day 1 and then testing performance on the tasks 24 hours later. Less disrupted sleep (expected for infants in the treatment group) was expected to be associated with higher performance on Day 2.

Specific Cognitive, Linguistic, and Social Domains and Associated Measures

The first year of life is marked by a number of cognitive, linguistic, and social achievements. Six developmental indicators in particular are related to long-term outcomes and thus are the targets for data collection for the proposed research. Additional measures may be obtained to assess sleep patterns and characterize the samples.

(1) Speed of Processing

The speed with which information is processed is a major determinant of cognitive achievement. There are age-related differences in processing speed and within an age group there are individual differences in speed of processing. Specifically, older children process information more rapidly than younger children (Kail, 1988) and within an age group, individuals with faster processing speed perform at higher levels on a number of experimental and standardized tasks (Rose, Feldman, & Jankowski, 2002). In the first year of life, processing speed is measured with a task that requires that infants encode the features of faces presented on a computer screen and then differentiate the encoded faces from others that look similar to them. Infants who encode the features more rapidly and also make finer discriminations among faces are identified as “fast processors.” Dependent measures include the length of time spent looking at the study faces; and during the test phase, the percentage of time spent looking at old versus new faces, the number of switches between study and test faces, and the duration of peak look to old and new faces.

(2) Attention

A critical cognitive achievement is the ability to regulate attention to the task at hand, thereby eliminating potential distractions. Early in the first year of life infants' attention is largely controlled by outside forces such as how attractive or brightly colored an object is. By the middle to the end of the first year, infants develop the ability to regulate their own attentional focus (Oakes & Tellinghuisen, 1994). Better attentional control is associated with cognitive and academic achievement (Colombo, 1993). In the first year, attention is measured with an object examining task. Infants are given eight 30 second periods to examine an object. They then are given two test trials, one with the same object and one with an object that differs from it on a small number of dimensions. Infants' behaviors during the examining and test phases are coded for evidence of attention to specific discriminating features. Dependent measures include the speed with which the infants familiarize to the object during the examination phase; and during the test phase, the duration of visual examining of the old and new stimuli, the duration of peak visual examination of stimuli, time spent mouthing the objects, and relations between examining with the eyes and examining with the mouth.

(3) Memory

The ability to remember is critical to learning and all manner of cognitive achievements. Throughout the first year of life, the length of time that infants remember and the amount of information that they are able to retain over time increase (Bauer, 2004). A task that is sensitive to both age-related and individual differences in memory early in life is elicited imitation. Infants are shown how to perform novel actions and sequences of actions using novel objects and then are permitted to imitate. Differences in behavior before and after exposure to the model are indicative of learning and memory. The task is sensitive to developmental delay associated with premature birth (de Haan, Bauer, Georgieff, & Nelson, 2000) and also is predictive of later performance on standardized measures of memory (DeBoer, Cheatham, Stark, & Bauer, 2005). Dependent measures include the number of individual target actions and the number of pairs of actions produced in the target order.

(4) Language Development

By the end of the first year of life, infants comprehend many words and some infants produce words. Even before they understand and produce speech, infants use and understand a variety of communicative gestures. The number of communicative gestures that they understand and use is predictive of later language development (Fenson et al., 1994). Because parents see their infants in many more contexts and over much longer periods of time than researchers can, valid and reliable parent report measures of early gestural and word use have been developed. The MacArthur-Bates Communicative Development Inventory: Words and Gestures is available in Mandarin and may provide a measure of achievement in the language domain. Dependent measures include the number of words and gestures understood and produced.

(5) Temperament

Infants differ with respect to their behaviors and reactions in everyday and novel situations. Some of the ways in which they differ have implications for social and cognitive development. For example, infants who are more sociable find it easier to approach novel people and objects and interact with them. These infants tend to perform at higher levels on memory tasks (Bauer, Burch, & Kleinknecht, 2002). Infants also vary in their ability to regulate their own behavioral states and emotional reactions. Better behavioral self regulation is associated with later moral development (Kochanska, Murray, & Coy, 1997) as well as academic achievement (Pintrich & De Groot, 1990). As in the case of language and gesture development, because parents see their infants in many more contexts and over much longer periods of time than researchers can, valid and reliable parent report measures of temperament have been developed. The Infant Behavior Questionnaire may be translated into Mandarin and may provide a measure of infant temperament. Dependent measures include parental ratings of behavior on each of the subscales of the questionnaire.

(6) Mother-Infant Communication

The relationship that an infant has with her or his mother is critical not only for the physical safety and security of the infant but for his or her psychological well being and later peer acceptance (Gunnar, 2001). Mother-infant dyads differ in the amount of synchrony they display during their face-to-face interactions. By noting the details of the facial behavior (looks to partner, looks away) and vocal behavior (baby vocalizes, mother calls baby's name) produced by mother and baby, and especially the interplay between these different behaviors, the level of behavioral synchrony within the dyad may be determined. These measures are an indication of early quality of nonverbal communication, which is in turn likely to affect infants' emotion regulation and emotional development more generally (Eckerman, Oehler, Hannan, & Molitor, 1995; Kaye & Fogel, 1980; Tronick, Cohn, & Shea, 1986). The mother-infant interaction and code the video record for visual and vocal behavior may be recorded, and conditional probabilities may be used to determine whether and how one partner influenced or was influenced by the other. Dependent measures include the number of bouts of shared attention, maternal strategies for engaging infants, the number of looks to partner and looks away, and the number vocalizations by mother and baby.

(7) Additional Measures

Throughout the study period, parents of enrolled infants may be asked to complete a sleep log or sleep questionnaires to establish sleeping and waking patterns. For the period between Sessions two and three (see below), infants may wear an actigraph (Actiwatch) to further characterize sleep patterns. Parents may be asked to complete a family demographics questionnaire to permit characterization of the samples.

Method

Participants may be 80 infants, half in a diaper treatment group and half in a no-treatment control group. Infants may be six months of age (range=5 months, 15 days-6 months, 15 days) at the time of enrollment in the study. Half may be female and half may be male. To be included in the study, infants may be required to have been born at term and to have had no major medical complications at birth and to have been experiencing an apparently normal course of development. In addition, infants may be selected into the study based on reported diapering practices. Specifically, infants whose parents reported that they may have had four (4) or more diaper changes per night may participate.

Procedure/Design

Infants may be enrolled at six months of age. One (1) week prior to the first visit to the laboratory, potential participants may be visited in their homes by a staff member. Parents may be interviewed to make certain their infants meet the eligibility criteria for participation. For infants in the diaper treatment group, during this visit staff members may educate the parents about the diaper treatment. The staff members may provide demonstrations and information about the diapers' absorbency and their effectiveness in keeping wetness away from the skin. They may make clear to the parents that changing the disposable diaper less frequently (or not at all) overnight may not jeopardize their baby's skin health. Importantly, the staff members also may make clear that it is the parents' decision how often to change their baby's diaper in the night.

The data collection may occur in four (4) sessions. Infants in the diaper treatment group and the no-treatment control group may receive identical testing procedures. The only difference between these groups is that the diaper treatment group wear PAMPERS® at night throughout the study period and the control group follow their typical nighttime routine with (presumably) more frequent diaper changes.

Session 1 may provide an opportunity to collect baseline measures for all infants on the Speed of Processing, Attention, and Memory tasks; for parents to fill out the Language Development and Temperament questionnaires; and to engage in the mother-infant communication task.

Session 2 may take place five (5) weeks later, and may consist of the “study phase” of each of the three cognitive tasks (Speed of Processing, Attention, and Memory). That is, in the Speed of Processing task, infants may be familiarized with the faces. In the Attention task, infants may receive eight 30 second trials of exposure to a familiarization stimulus. In the Memory task, infants may watch as an experimenter models three 2-step sequences enacted with props. Once the study phases are complete, parents may be introduced to the Actiwatch and instructed to use it to record their infants' movements during sleep that night to provide a measure of frequency and length of sleep disruption as well as some indication of sleep stages. Parents and infants then may be dismissed from the laboratory.

Session 3 may occur one (1) day (24 hours) after Session 2. It may consist of the “test phase” of the three cognitive measures. That is, in the Speed of Processing task, infants may receive test trials to determine whether they discriminate the familiarization from the test stimuli. In the Attention task, they may receive one (1) trial with the familiarization stimulus and one (1) trial with a novel stimulus to determine patterns of attention. In the Memory task, infants may be presented with the stimuli for each of the three sequences in turn to determine if they remembered the actions modeled the previous day.

Session 4 may occur approximately one (1) week after Session 3 and may consist of the same procedures as Session 1.

For the first week of the study, families of infants in the diaper treatment group may be visited in the home three times to answer questions the parents have about the diapers, and to check for compliance with use of the diapers. For the remaining five (5) weeks of the study, families of infants in the diaper treatment group may be visited one (1) time per week to deliver the next week's supply of diapers, to answer any questions the parents have about the diapers, and to check for compliance with use of the diapers.

Analysis

This design may allow for multiple levels of comparisons between the diaper treatment and no-treatment control groups. To test for general gains associated with establishment of more regular sleep patterns, performance of the groups at Sessions 1 and 4 may be compared. The groups may not be expected to differ at Session 1 (before introduction of the treatment) but may be expected to differ at Session 4, with higher performance among infants in the diaper treatment group. To test for relations between sleep and consolidation performance of the groups at Session 3 may be compared. The groups may be expected to differ on the tests, with higher performance among infants in the diaper treatment group. For both questions, the sleep logs/questionnaires may be used to determine whether there is a dose/response effect such that infants with fewer nighttime awakenings performance at higher levels. In addition, the actigraph data may be used to determine whether sleep patterns between study and test relate to performance in systematic ways.

Example Analysis System 5

In the disclosed analysis system, over a 6-week period, the sleep patterns of a group of Chinese infants may be modified by offering their caregivers the option of using a high absorbency disposable diaper, instead of their traditional cloth diapers. Cloth diapers typically require multiple interruptions of infants' nocturnal sleep for the purpose of diaper change, whereas high absorbency disposable diapers do not. The effect of this manipulation may be investigated on infant sleep habits and on the development of temperament using caregiver-report measures obtained before starting and after completing the six-week sleep intervention.

There is an extensive literature supporting clear relations between nighttime sleep habits and temperament. For example, in a normative sample, the amount of sleep obtained at night by American infants was negatively related to mood, adaptability, rhythmicity, and approach (Weissbluth, 1981). Similar findings resulted from a cross-cultural comparison of Chinese-American and non-Chinese infants. Weissbluth (1982) asked parents to rate the temperament of their infants on the Carey Infant Temperament Questionnaire (Carey & McDevitt, 1978) and report on the nighttime sleep habits of their infants. The parents of Chinese-American infants rated the temperament of their infants as more difficult relative to non-Chinese infants, such that they were more withdrawn, less adaptable, and more negative in mood. Notably, Chinese-American infants also slept less at night relative to non-Chinese infants. These findings are similar to those obtained from infants and children with sleep problems, which indicate that individuals with difficult temperament profiles (characterized by difficulty approaching and adapting to novel situations as well as intense negative reactions; Carey, 1970) are more likely to experience nighttime sleep problems relative to those with easy temperaments (characterized by easy approach and adaptation to the new as well as mild, positive mannerisms; Carey, 1978) (Weissbluth, Davis, & Poncher, 1984; Weissbluth & Liu, 1983). Taken together, these data indicate that sleep and temperament are related. However, because none of the published studies has included experimental manipulation of nighttime sleep habits, the work does not indicate whether sleep problems cause infants to develop difficult temperaments, or whether having a difficult temperament predisposes infants to sleep problems.

A better understanding of the relation between sleep and temperament may be clinically important, in that early interventions may be constructed to target infants with sleep problems so as to suppress the development of difficult temperaments over time or to identify individuals with presumed vulnerability to nighttime sleep disorders based on their temperamental profiles. In addition, understanding the influence of sleep habits on temperament may provide some insight into cultural differences in temperament, given that infant sleep habits vary widely as a function of societal beliefs and cultural norms (Lozoff & Brittenham, 1979). Although sleep habits and temperament in infancy are both determined in a multiple manner, systematic investigations designed to understand the contribution of various experiential factors to the development of temperament have yet to be conducted by conventional analysis systems. Such investigations are worthwhile, in that differences in various temperament dimensions and in the structure of temperament have been observed in infants and children tested across the globe. For instance, Hsu, Soong, Stigler, Hong, and Liang (1981) indicated that, relative to American infants, Chinese infants tested in Taiwan were more intense and more negative in mood. They also were less active and rhythmic, less likely to approach novel items, less adaptable and distractible, and had lower thresholds of responsiveness. Using the Infant Behavior Questionnaire (IBQ; Rothbart, 1981), Gartstein et al. (2006) compared the temperamental profiles of Chinese, Spanish, and American infants tested in the first year of life. Chinese infants were rated as more active and more prone to distress to limitations than were American or Spanish infants. In addition, Chinese infants were rated as more fearful than American infants. The authors suggest that these findings may relate to differences in Eastern versus Western cultural values that may affect parenting attitudes and practices. For example, they speculated that the cultural emphasis placed on introverted or inhibited personality traits in China, perhaps resulting from the influence of Confucianism, may have resulted in greater amounts of fearfulness in Chinese infants. These authors also suggest that although early differences in temperament may predispose infants to psychopathology later in life, long-term functional outcomes may be mediated by cultural beliefs and values (Draguns & Tanaka-Matsumi, 2003). Notably, differential behavioral outcomes may also partially result from cross-cultural genetic variability as well as uninvestigated interactions between specific genetic profiles and cultural experiences (see Krueger, Johnson, & Kling, 2006, for a review on behavior genetics and temperament).

One clear example of similar temperamental profiles resulting in different functional outcomes largely as a function of societal factors and parental beliefs and attitudes is in the case of behavioral inhibition (see Chen, Wang, & DeSouza, 2006, for a review). Chinese toddlers are shier and more behaviorally inhibited relative to Canadian toddlers (Chen et al., 1998), and Chinese children manifest higher levels of negative affect during the school years relative to American children (Ahadi, Rothbart, & Ye, 1993). In China, however, such traits are perceived as positive and are encouraged, such that shy and quiet children are praised for their good behavior. These children are viewed as interactively competent, they engage in positive social relationships, and they do not manifest the difficulties experienced by American children with the same behavioral profiles (Chen et al., 2006; Ho, 1986; Ho & Kang, 1984; Rubin, 1998; Rubin, Burgess, & Coplan, 2002), including negative feelings about the self, peer rejection, loneliness, depression, and persisting deficits in social functioning (Boivin, Hymel, & Bukowski, 1995; Rubin, Chen, McDougall, Bowker, & McKinnon, 1995). To summarize, these data indicate that cultural beliefs and societal norms impact the types of experiences infants and children are expected to encounter as well as the reactions of those around them. These experiences and norms are then internalized and contribute to long-term functional outcomes.

In the disclosed analysis system, caregiver ratings of infant temperament may be assessed before and after a manipulation of infant sleep habits in China. China offered a special opportunity to conduct this work because there is a traditional and prevailing custom in the care of infants to use a type of cloth diaper not intended to absorb or retain waste for extended periods and which, therefore, requires frequent disruptions of the infant's nocturnal sleep period, for the purpose of diaper changes. By familiarizing families with the absorbency and other attributes of a high quality disposable diaper, there may be an inability to affect the number of nighttime sleep disruptions the infants experienced, compared to infants who remained on the traditional cloth diaper.

Infants may be enrolled in the study of the analysis system at six months of age; the diaper manipulation may last six weeks. The age of six months may be selected as optimal for other of the assessments obtained in the study, but may not be included in this report, including attention, memory, speed of processing, and mother-infant dyadic interaction. For purposes of the manipulation, caregivers may be randomly selected to continue using traditional cloth diapers or to use disposable diapers. Caregivers who may be selected to use disposable diapers may also be asked to avoid waking their infants for diaper changes or other reasons, thereby allowing the infants the best opportunity for uninterrupted nights of sleep. Caregivers of infants who wore cloth diapers may not be provided with any instructions regarding the need to suspend nighttime disruptions or reduce their frequency. Alternatively, caregivers may be provided with the same instructions, or other instructions. The intervention for a 6-week period may be carried out to allow caregivers the opportunity to gain familiarity with and become comfortable with disposable diaper application and use, to promote the development of stable sleep patterns resulting from the use of disposable diapers, and to allow for investigation of the long-term effects of disposable diaper use on cognitive and social-emotional outcomes. Because previous experimental studies of this nature have not been conducted, a specific hypotheses may not be formulated regarding the influence of sleep habits on temperament, except to indicate that altered sleep habits may impact caregiver perceptions of infant behavior.

Method

Participants

Eighty-two six-month-old infants (mean age=6 months, 2 days; range 5 months, 13 days to 6 months, 18 days; 39 girls and 74% co-sleepers) participated in the study. An additional three infants were enrolled but did not return for all of their visits to the test site. Infant participants were enrolled by a paid organization from the cities of Jinzhou (n=43 infants; 20 girls) and Shenyang (n=39 infants; 19 girls) in the Liaoning province of northeastern China. Shenyang and Jinzhou are cities of comparable size (5 million and 3 million, respectively) with similar industries and socio-economic status. They were thus expected to be similar in regard to childrearing practices and cultural values.

Potential participants were contacted using hospital birth records and were invited to participate if they met the following criteria: full-term birth (40±4 weeks gestational age); were experiencing an apparently normal course of development with no known mental or physical conditions or disorders; routinely wore cloth diapers, and wore them exclusively during the seven nights prior to the recruitment interview; were disturbed at least four times per night for infant toilet training or other purposes; and caregivers were willing to suspend nighttime waking procedures if requested to do so by study protocol. Only infants of caregivers who answered all questions affirmatively were enrolled as participants. Infants were excluded from participation if their caregivers indicated that they had experienced any perinatal complications; if they had previously participated in a study involving the use of disposable diapers; or if they had chronic sleep conditions such as apnea, reflux, or colic. All of the infants were of Chinese descent; most were of Han ethnicity, which is the predominant ethnic group in China. Caregivers of infant participants were provided with free transportation to and from the test site as well as monetary incentives or infant toys in appreciation for their participation. Although incentives varied by city of enrollment, at both sites, the combined value of the monetary incentives and infant toys was approximately $75. In addition, consistency in cultural beliefs and values may be maintained by testing infants in the same region of China at the same time. In this way, the influence of nighttime sleep habits on caregiver perceptions of infant behavior may be examined while ensuring that cultural beliefs and values may be held constant across the different groups in the sample.

Materials

Caregivers of infants assigned to wear disposable diapers during the 6-week diaper manipulation were provided with free supplies of PAMPERS BABY DRY® (The Procter & Gamble Co, USA), a high absorbency disposable diaper commercially available in China. Cloth diapers were not given to caregivers of infants enrolled in the cloth diaper control group.

Measures and Procedures

Group assignment. At the time of enrollment in the study, infants were assigned to participate in either the cloth diaper control group or the disposable diaper group on a semi-random basis. Specifically, the first enrolled infants of each sex were assigned to the cloth diaper control group; the next infants of each sex were assigned to the disposable diaper group; and so on, until the complete sample of infants was realized. The enrollment procedure is semi-random in that although infants were assigned to groups based on a fixed alternating schedule, families were scheduled to arrive for their enrollment interviews in no particular order. At the end of the enrollment interview, caregivers of infant participants assigned to the disposable diaper group were given one bag of disposable diapers with instructions to use them during daytime hours only as a means of familiarizing themselves and gaining practice with disposable diaper application and use.

Infant Behavior Questionnaire-Revised (IBQ-R): First administration. At the end of the enrollment interview, caregivers of all infant participants were given a blank version of the Infant Behavior Questionnaire-Revised (IBQ-R; Gartstein & Rothbart, 2003) and were asked to return the completed questionnaire at their first visit to the test site the next week. The questionnaire was translated to Mandarin by one native-speaking bilingual Chinese-American and back-translated by another. It was then checked for dialectical coherence and accuracy by study personnel in China. The IBQ-R is a 191-question parent-report instrument that includes fourteen temperament dimensions shown in Table 8. The IBQ-R was constructed to measure individual differences in temperament and also allows for examination of the development of temperament in infancy as well as elucidation of the contribution of experiential factors to various temperamental profiles. The IBQ-R is among the best caregiver-report measure available, in that individuals are asked to report on easily observable behaviors pertaining to recently occurring events. The inventory is an extension of the IBQ (Rothbart, 1981) and was created using operational definitions for each temperament dimension as well as item-specific and conceptual analyses to ensure internal validity and minimize conceptual overlap among the scales (Gartstein & Rothbart, 2003).

Caregivers of infant participants completed the IBQ-R as instructed, circling their responses on a 7-point Likert-type scale to indicate the frequency with which their infants reacted in specific ways over the seven days prior to its receipt. Upon return of the questionnaire, study personnel reviewed each inventory to make sure that all questions were answered. When missing responses were noted, study personnel answered any questions caregivers had about the unanswered item before asking them to provide a response. At that time, caregivers could choose to provide an answer on the 7-point scale or to indicate that the question did not pertain to the experiences of their infant. Caregivers did not report any difficulty in responding to the questions on the IBQ-R.

The diaper manipulation. The diaper manipulation commenced upon return of the questionnaire. At that time, caregivers of infant participants in the disposable diaper group were given a live demonstration of how to apply the product and were informed of the benefits of its use (e.g., that the product they were given was more absorbent and less prone to leakage, for which reasons infants need not be disrupted as often during the night for diaper changes). Caregivers were then instructed to use disposable diapers on their infants each and every night for the following 6 weeks and to avoid waking their infants for diaper changes or toilet training practices as much as possible, thereby allowing the infants the best opportunity for uninterrupted nights of sleep. Caregivers of infants in the cloth diaper control group were not provided with any direction regarding the need to suspend nighttime disruptions or minimize their frequency. Alternatively, caregivers may be provided with the same instructions, or other instructions.

Sleep inventories and diaries. Study personnel visited the homes of enrolled families fifteen times throughout the 6-week intervention. One visit was completed before the start of the diaper manipulation to obtain a baseline measure of how often the infants were disturbed the previous night for diaper changes or other reasons. The other fourteen visits occurred during the 6-week diaper manipulation. At these visits, study personnel asked caregivers to report whether they placed their infant in the appropriate type of diaper the previous night and how many times the infants' sleep was disrupted for diaper changes or other reasons. For families of infants in the disposable diaper group, study personnel also provided additional free supplies of disposable diapers and reinforced the importance of reducing the number of times infants were disrupted during the night, either for diaper changes or other reasons. Other examples, of sleep evaluation include using any other time variants so that a test duration and one or more time specified evaluation is taken prior to the end of the test duration.

Caregivers also completed detailed nighttime sleep diaries for three consecutive nights during either the fourth or fifth weeks of the diaper manipulation. Caregivers were asked to indicate when the infant was put to bed at night and when the infant woke the next morning; the number of times the infant's sleep was disrupted at night, and whether the infant woke up as a result of the disruption; and the duration of time during which the infant's sleep was disrupted, again differentiating between when the infant was and was not woken from sleep. Caregivers were asked to record each event immediately after its occurrence, thereby allowing for the most accurate self-report measure possible.

Infant Behavior Questionnaire-Revised (IBQ-R): Second administration. During the fifth week of the of the diaper manipulation, caregivers of infant participants were given a blank version of the IBQ-R and were asked to return the completed questionnaire to the test site the following week. Requested disposable diaper use for the purposes of maintaining study protocol ceased after the questionnaires were collected.

Data Reduction

Sleep inventories and diaries. Caregiver report of infant sleep habits at the fifteen home visits allowed for the creation of variables pertaining to the frequency with which caregivers disturbed their infants over the fourteen queried nights, including one night before the initiation of the diaper manipulation. After the start of the diaper manipulation, caregivers also reported the number of times they placed their infants in the appropriate type of diaper at night as well as the frequency with which they disturbed their infants for diaper changes or other reasons.

The more detailed information recorded in the nighttime sleep diaries allowed for the creation of duration and frequency variables pertaining to sleep habits over three consecutive nights during either the fourth or fifth weeks of the diaper manipulation. Duration measures included the cumulative amount of time infants were disturbed, the amount of time infants were disturbed and woken from sleep, and the amount of time infants were disturbed but not woken from sleep; duration measures were averaged across the three reported nights. Frequency measures included the cumulative number of times infants were disturbed, the number of times infants were disturbed and woken from sleep, and the number of times infants were disturbed and but not woken from sleep; as with the duration measures, mean values were created by averaging responses across the three reported nights.

IBQ-R. The temperament data obtained from the IBQ-R were reduced into the fourteen scales shown in Table 8. Scales were computed separately for the first and second administrations of the questionnaire.

TABLE 8
Names and Descriptions of Scales on the IBQ-R (Definitions Obtained from Gartstein
& Rothbart, 2003)

| Scale Name | Description |
|---|---|
| Activity level | Gross motor activity, including movement of arms and legs, squirming and |
| | locomotor activity |
| Approach | Rapid approach, excitement, and anticipation of pleasurable activities |
| Cuddliness | Expression of enjoyment and molding of the body to being held by a |
| | caregiver |
| Distress to limitations | Fussing, crying, or showing distress while (a) in a confining place or |
| | position; (b) in caretaking activities; (c) unable to perform a desired action |
| Duration of orienting | Attention to and/or interaction with a single object for extended periods of |
| | time |
| Fear | Startle or stress to sudden changes in stimulation, novel physical objects, or |
| | social stimuli; inhibited approach to novelty |
| High-intensity pleasure | Pleasure or enjoyment related to a high stimulus intensity, rate, complexity, |
| | novelty, and incongruity |
| Low-intensity pleasure | Amount of pleasure or enjoyment related to a high stimulus intensity, rate, |
| | complexity, novelty, and incongruity |
| Perceptual sensitivity | Detection of slight, low-intensity stimuli from the external environment |
| Rate of recovery from | Rate of recovery from peak distress, excitement, or general arousal; ease of |
| distress | falling asleep |
| Sadness | Lowered mood and ability related to personal suffering, physical state, |
| | object loss, or inability to perform a desired action; general low mood |
| Smiling and laughter | Smiling and laughter during general caretaking or play |
| Soothability | Reduction of fussing, crying, or distress when soothing techniques are used |
| | by the caregiver |
| Vocal reactivity | Amount of vocalization exhibited by the baby in daily activities |

Results

Sensitivity to several potential influences on the development of temperament in infancy, including infant's sex, family sleeping practices (independent sleeper or co-sleeper), and regional differences in sleeping habits may be included. These variables were included as categorical covariates in some analyses. In between-subjects analyses, statistically non-significant covariates were removed by successively eliminating the most non-significant variables until the model contained only covariates that were significant at p≦0.10. Correlations were conducted controlling for each of these three variables. Corrections were not made for multiple comparisons because of the exploratory nature of the investigation and following Rothman's argument (1990) that all obtained results be reported, with meaningful patterns interpreted further and left for corroboration through replication. Significant and marginal effects are reported when p≦0.10. Measures of internal consistency for separate administrations of the IBQ-R across groups are shown in Table 9.

TABLE 9
Measures of Internal Consistency for Separate Administrations
of the IBQ-R Across Groups

| Behavioral Scale | First Administration | Second Administration |
|---|---|---|
| Activity level | .70 | .64 |
| Approach | .78 | .81 |
| Cuddliness | .75 | .82 |
| Distress to limitations | .82 | .80 |
| Duration of orienting | .80 | .83 |
| Fear | .88 | .91 |
| High intensity pleasure | .69 | .68 |
| Low intensity pleasure | .84 | .85 |
| Perceptual sensitivity | .85 | .85 |
| Rate of recovery from | .76 | .74 |
| distress | | |
| Sadness | .80 | .83 |
| Smiling and laughter | .73 | .71 |
| Soothability | .71 | .79 |
| Vocal Reactivity | .69 | .75 |

Sleep Inventories and Diaries

Investigation of relations between group assignment and infant sleep habits as measured at the home visits using SAS PROC GLM with group as a between-subjects factor may be made. As shown in Panel A of Table 10, before the initiation of the diaper manipulation, the sleep of infants enrolled in the cloth diaper control and disposable diaper groups was disrupted a similar number of times at night. The groups also experienced a similar number of diaper changes, and similar numbers of all other distuptions. During the diaper manipulation, however, the sleep of infants in the cloth diaper control group was disrupted a greater total number of times during the night, F(1, 80)=332.43, p<0.0001, d=4.03, and a greater number of times for diaper changes, F(1, 79)=591.07, p<0.0001, d=5.30, relative to infants in the disposable diaper group. There was no difference in the number of disruptions not including diaper changes. In addition, caregivers reported placing their infant in the appropriate type of diaper 100% of the nights under investigation. Therefore, the apparent differences in infant sleeping habits likely resulted from the imposed diaper manipulation, as group differences were only observed for dependent measures that included assessment of diaper changes.

Also, nighttime sleep diaries may be used between-subjects analyses to examine sleep habits. Because between-subjects analyses revealed the same pattern of results for caregivers who reported on their infant's sleep habits during the fourth and fifth weeks of the diaper manipulation, the data for analyses may be combined. A first analysis was conducted to determine whether infants spent similar amounts of time in bed at night, independent of group assignment. As shown in Panel B of Table 10, infants in the cloth diaper control group spent significantly more time in bed before accounting for any nighttime disruptions relative to infants who wore disposable diapers: F(1, 79)=16.07, p<0.0001, d=0.86. This systematic group difference has the potential to bias the obtained pattern of results because of its inherent relation with the other dependent measures. Specifically, infants who spent more time in bed at night had greater opportunity for their sleep to be disrupted, both in terms of duration and frequency. Because the direction of the group difference supported our predictions, the amount of time spent in bed at night may be statistically controlled for by including this variable as a continuous covariate in all analyses involving measures from the nighttime sleep diaries.

TABLE 10
Nighttime Sleep Habits as Assessed at the Home Visits (Panel A) and
Nighttime Sleep Habits as Assessed by Sleep Diairies

| | | |
|---|---|---|
| | Cloth | Disposable |
| Sleep Measure | Diaper Control | Diapers |

Panel A: Nighttime Sleep Habits as Assessed at the Home Visits

| | | |
|---|---|---|
| Total number of disruptions at baseline | 5.16 ± .21 | 4.74 ± .21 |
| Number of diaper changes | 4.12 ± .19 | 3.93 ± .19 |
| All other disruptions | 1.04 ± .22 | .82 ± .22 |
| Total number of disruptions during | 70.98 ± 1.82 | 23.98 ± 1.82 |
| manipulation | | |
| Number of diaper changes | 51.05 ± 1.42 | 2.34 ± 1.42 |
| All other disruptions | 19.93 ± 2.43 | 21.63 ± 2.43 |

Panel B: Nighttime Sleep Habits as Assessed by Sleep Diairies

| | | |
|---|---|---|
| Total amount of time spent in bed at | 658.21 ± 9.55 | 604.07 ± 9.55 |
| night (in min.) | | |
| Total number of disruptions | 4.99 ± .14 | 2.07 ± .14 |
| Number of disruptions with wakes | 3.68 ± .18 | 1.68 ± .18 |
| Number of disruptions without wakes | 1.30 ± .13 | .41 ± .13 |
| Total time spent disrupted (in min.) | 84.94 ± 6.02 | 49.64 ± 6.02 |
| Time spent disrupted and awake | 73.04 ± 6.36 | 45.43 ± 6.36 |
| Time spent disrupted but not awake | 11.65 ± 1.64 | 4.45 ± 1.64 |
| Total amount of time spent in bed at | 563.91 ± 8.49 | 563.79 ± 8.49 |
| night after accounting for all | | |
| disruptions (in min.) | | |

Other data obtained from the nighttime sleep diaries indicated that the diaper manipulation was effective in producing variability in infant sleep habits. As shown in Panel B of Table 10, the sleep of infants in the cloth diaper control group was disrupted a greater total number of times, F(1, 80)=199.58, p<0.0001, d=3.12, and a greater number oftimes when accounting for disruptions that did and did not wake the infant from sleep: F(1, 79)=56.69, p<0.0001, d=1.05, and F(1, 78)=20.45, p<0.0001, d=0.92, respectively. In addition, the sleep infants who wore cloth diapers was disrupted a greater total amount of time, F(1, 79)=15.82, p<0.0002, d=1.27, and a greater amount of time when accounting for disruptions that did and did not wake the infant from sleep: F(1, 79)=8.68, p<0.004, d=1.04, and F(1, 78)=9.54, p<d=0.66, respectively. There was no difference in the amount of time infants spent in bed at night after accounting for all nighttime disruptions. Taken together, these data indicate that the diaper manipulation was effective in producing variability in the quality of infant sleep. Although infants spent similar amounts of time in bed at night after accounting for all nighttime disruptions, infants who wore disposable diapers experienced less fragmented sleep relative to infants who wore cloth diapers. That is, they experienced sleep disruptions fewer times at night and for a lesser amount of time, which allowed them greater opportunity for uninterrupted nights of sleep.1

Nighttime Sleeping Habits and Infant Temperament

Differences in temperament may be analyzed separately by administration of the IBQ-R to determine whether there were any a priori differences in temperament at the first administration and to identify whether the diaper manipulation differentially affected caregiver ratings of infant temperament at the second administration. These data are shown in Table 11. To capitalize on the variability in sleep habits and temperament in the complete sample, computed correlations may also be made between measures from the nighttime sleep diaries and the scales from the second administration of the IBQ-R. These correlations are shown in Table 12.

TABLE 11
Group Differences in Behavior at the First and Second Administrations of
the IBQ-R

| | First Administration | | Second Administration | |
|---|---|---|---|---|
| Behavioral | Cloth Diaper | Disposable | Cloth Diaper | Disposable |
| Scale | Control | Diaper | Control | Diaper |
| Activity level | 4.51 ± .11 | 4.47 ± .11 | 4.78 ± .10 | 4.76 ± .10 |
| Approach | 5.11 ± .13 | 4.97 ± .13 | 5.38 ± .12 | 5.05 ± .12 |
| Cuddliness | 5.54 ± .10 | 5.40 ± .10 | 5.29 ± .11 | 5.22 ± .11 |
| Distress to | 3.63 ± .13 | 3.51 ± .13 | 3.89 ± .13 | 3.66 ± .13 |
| limitations | | | | |
| Duration of | 3.79 ± .15 | 3.73 ± .15 | 3.81 ± .15 | 4.05 ± .15 |
| orienting | | | | |
| Fear | 3.42 ± .17 | 3.59 ± .17 | 3.38 ± .17 | 3.57 ± .17 |
| High intensity | 4.92 ± .12 | 4.96 ± .12 | 4.98 ± .12 | 5.03 ± .12 |
| pleasure | | | | |
| Low intensity | 5.01 ± .13 | 4.80 ± .13 | 4.65 ± .14 | 4.81 ± .14 |
| pleasure | | | | |
| Perceptual | 3.90 ± .20 | 3.89 ± .20 | 4.17 ± .16 | 4.35 ± .16 |
| sensitivity | | | | |
| Rate of | 4.79 ± .12 | 5.00 ± .12 | 4.85 ± .11 | 5.14 ± .11 |
| recovery from | | | | |
| distress | | | | |
| Sadness | 3.19 ± .14 | 3.15 ± .14 | 3.43 ± .14 | 3.25 ± .14 |
| Smiling and | 3.85 ± .15 | 4.02 ± .15 | 4.04 ± .16 | 3.91 ± .16 |
| laughter | | | | |
| Soothability | 4.99 ± .09 | 4.96 ± .09 | 5.01 ± .11 | 4.91 ± .11 |
| Vocal | 4.02 ± .14 | 4.04 ± .14 | 4.23 ± .13 | 4.36 ± .13 |
| reactivity | | | | |

TABLE 12
Correlations Between Measures of Nighttime Sleep Habits and Caregiver Ratings of
Infant Temperament

| | Duration Measures | | | Frequency Measures | | |
|---|---|---|---|---|---|---|
| | | Disrupted | Disrupted | | Disrupted | Disrupted |
| | | and | Not | Total | and | Not |
| Behavioral Scale | Total Time | Awake | Awake | Number | Awake | Awake |
| Activity level | .20^{□} | .16 | .15 | .22* | .16 | .12 |
| Approach | −.03 | −.01 | −.07 | .19^{□} | .24* | −.06 |
| Cuddliness | −.06 | −.09 | .11 | −.01 | .02 | −.04 |
| Distress to | .12 | .06 | .22* | .26* | .18 | .15 |
| limitations | | | | | | |
| Duration of | −.13 | −.12 | −.02 | −.15 | −.12 | −.07 |
| orienting | | | | | | |
| Fear | −.01 | −.03 | .06 | −.02 | .03 | −.08 |
| High intensity | −.04 | −.05 | .04 | .08 | .07 | .03 |
| pleasure | | | | | | |
| Low intensity | −.14 | −.12 | −.07 | −.08 | .00 | −.14 |
| pleasure | | | | | | |
| Perceptual | −.11 | −.05 | −.21^{□} | −.08 | .04 | −.20^{□} |
| sensitivity | | | | | | |
| Rate of recovery | −.26* | −.19^{□} | −.29* | −.26* | −.11 | −.27* |
| from distress | | | | | | |
| Sadness | −.03 | −.06 | .11 | .08 | .01 | .12 |
| Smiling and | −.11 | −.09 | −.09 | .15 | .20^{□} | −.05 |
| laughter | | | | | | |
| Soothability | −.05 | −.06 | .02 | .03 | .03 | .01 |
| Vocal reactivity | −.15 | −.15 | −.03 | −.03 | .03 | −.10 |

Note:
Significant and marginal effects are indicated as follows:
*p ≦ .05,
^{□}p ≦ .10

Group differences in infant temperament at the first administration of the IBQ-R. To determine whether infant temperament differed as a function of group assignment before the start of the diaper manipulation, between-subjects analyses on each subscale obtained from the first administration of the IBQ-R may be conducted. Group differences were not found on any of the subscales.

Group differences in infant temperament at the second administration of the IBQ-R. To determine whether the diaper manipulation impacted caregiver ratings of infant temperament, between-subjects analyses on each subscale obtained from the second administration of the IBQ-R may be conducted. Marginal group differences were apparent on two subscales. Infants infants who wore disposable diapers received lower ratings on the approach scale relative to infants who wore cloth control diapers: F(1, 80)=3.79, p=0.06, d=0.42. Conversely, infants in the disposable diaper group received higher ratings on the scale of rate of recovery from distress relative to infants who wore cloth control diapers: F(1, 80)=3.23, p=0.08, d=0.40. Taken together, these data indicate that the diaper manipulation produced noticeable effects on caregiver ratings of infant temperament after a 6-week sleep intervention in the absence of any a priori group differences.

Correlational analyses. Correlations between frequency and duration measures obtained from the sleep diaries and caregiver ratings of infant temperament obtained from the second administration of the IBQ-R indicated that infant sleep habits significantly predicted caregiver ratings of temperament at the end of the diaper manipulation. Not computed may be correlations between measures of temperament obtained from the first administration of the IBQ-R and nighttime sleep habits because the offset in the timing of measurements would make interpretation difficult, as sleep habits (Acebo et al., 2005; Louis, Cannard, Bastuji, & Challamel, 1997) and temperament (Rothbart, 1994, 1988, 1986) both undergo substantial development in the first year of life and beyond.

Positive correlations were found between measures of nighttime sleep habits and caregiver ratings of infant temperament at the second administration of the IBQ-R on the scales of activity level, approach, distress to limitations, and smiling and laughter. As shown in Table 12, caregiver ratings of infant activity level were marginally related to the total amount of time infants' sleep was disrupted at night and were significantly related to the total number of times infants' sleep was disrupted at night. There was a marginal positive correlation between approach and the total number of times infants' sleep was disrupted at night, as well as a significant positive correlation with the total number of times infants' sleep was disrupted at night when infants woke from sleep. Significant relations were also found between distress to limitations and (a) the amount of time infants' sleep was disrupted when infants did not wake, as well as (b) with the total number of times infants' sleep was disrupted at night. A marginal positive relation was found between smiling and laughter and the number of times infants' sleep was disrupted and infants woke from sleep. In summary, more disrupted nighttime sleep was related to higher caregiver ratings of activity level, the approach or anticipation of pleasurable activities, distress when placed in limiting situations, and smiling and laughter.

Negative correlations were found between measures of nighttime sleep habits and caregiver ratings of infant temperament on the scales of perceptual sensitivity and rate of recovery from distress. Specifically, there were marginal negative correlations between perceptual sensitivity and the number and duration of times infants' sleep was disrupted when they were not woken from sleep. In addition, there were negative relations between rate of recovery from distress and (a) the total amount of time infants' sleep was disrupted at night, (b) the amount of time infants' sleep was disrupted when they did not wake from sleep, (c) the total number of times infants' sleep was disrupted at night, and (d) the number of times infants' sleep was disrupted when they did not wake from sleep. A marginal negative correlation was also found between rate of recovery from distress and the amount of time infants' sleep was disrupted when they were woken from sleep. Thus, more disrupted nighttime sleep was related to lower ratings on perceptual sensitivity and rate of recovery from distress.

There were no significant or marginal correlations between nighttime sleep habits and caregiver ratings from the second administration of the IBQ-R on any of the other subscales.

Developmental Change and Stability in the Temperament of Chinese Infants

Administration of the IBQ-R both before the initiation of and at the end of the diaper manipulation allowed for investigation of developmental change and stability in the temperament of Chinese infants with traditional sleeping habits as well as for an examination of the influence of modified sleeping habits on the typical pattern of development. Developmental change may be examined by subtracting caregiver ratings obtained from the first administration of the questionnaire from caregiver ratings obtained from the second administration. Between-subjects analyses may be conducted to determine whether the amount of change differed as a function of group assignment. Stability may be examined by comparing caregiver ratings of infant temperament across the two administrations of the questionnaire separately by group. Correlations were computed separately by group across administrations of the IBQ-R and were statistically compared to determine whether differential stability was apparent in infancy as a function of group assignment. When paired in this manner, these analyses allow for investigation of the development of temperament in Chinese infants who wore traditional cloth diapers as well as in those who wore disposable diapers, which allows for insight as to how the diaper manipulation altered the characteristic pattern of development.

Analyses of change. Caregiver ratings increased significantly across the two administrations of the IBQ-R on the subscale of activity level for infants who wore disposable diapers, F(1, 79)=3.84, p=0.05, d=0.42, and marginally for infants who wore were enrolled in the cloth diaper control group, F(1, 78)=3.51, p=0.06, d=0.31 (see Table 12). A marginal decrease in low intensity pleasure was found for infants who wore cloth control diapers, F(1, 79)=3.71, p=0.06, d=0.40, whereas a marginal increase in perceptual sensitivity was found over time for infants who wore disposable diapers, F(1, 80)=3.77, p=0.06, d=0.43. Between-subjects analyses conducted using change scores indicated that the groups experienced differential rates of change on low-intensity pleasure, F(1, 80)=4.17, p<0.04, d=0.45, such that infants who wore disposable diapers increased in low-intensity pleasure over time relative to infants who wore cloth control diapers.

Analyses of stability. The stability of temperament may be investigated over time by group by correlating each scale of the IBQ-R across administrations. As shown in Table 13, the cloth diaper control group showed nominally higher correlations on ten of the fourteen temperament scales; marginal differences were obtained on the subscales of approach and high intensity pleasure. These data suggest that greater stability over time was found for infants in the cloth diaper control group, thereby indicating that the diaper manipulation directly impacted the developmental stability of temperament. A greater number of significant or marginal differences likely were not found due to the relatively small sample size across groups.

TABLE 13
Change Scores across Administrations of the IBQ-R by Group

| | Group | |
|---|---|---|
| Behavioral Scale | Cloth Diaper Control | Disposable Diaper |
| Activity level | .27⁺ ± .10 | .29* ± .10 |
| Approach | .26 ± .12 | .09 ± .12 |
| Cuddliness | −.25 ± .08 | −.18 ± .08 |
| Distress to limitations | .26 ± .09 | .15 ± .09 |
| Duration of orienting | .02 ± .16 | .32 ± .16 |
| Fear | −.04 ± .17 | −.01 ± .17 |
| High intensity pleasure | .07 ± .12 | .07 ± .12 |
| Low intensity pleasure | −.36⁺ ± .13 | .02 ± .13 |
| Perceptual sensitivity | .27 ± .17 | .45⁺ ± .17 |
| Rate of recovery from | .05 ± .10 | .13 ± .10 |
| distress | | |
| Sadness | .24 ± .14 | .10 ± .14 |
| Smiling and laughter | .19 ± .13 | −.11 ± .13 |
| Soothability | .02 ± .11 | −.05 ± .11 |
| Vocal reactivity | .20 ± .12 | .32 ± .12 |

Note:
All of the correlations for the individual scales of the IBQ-R are significant, p < .05. Significant and marginal differences between the groups are indicated as follows: ^{□} p ≦ .10.

Discussion

One of the primary motivations for the present investigation was to determine whether sleep habits related to manifestations of temperament in Chinese infants. Previous research has indicated that patterns of temperament vary across cultures (Ahadi et al., 1993; Gartstein et al., 2006; Gartstein, Slobodskaya, & Kinscht, 2003; Hsu et al., 1981), even during the neonatal period (Freedman & Freedman, 1969). Research has not yet established, however, which specific environmental factors might influence the development of temperament in infancy. Such studies are difficult to conduct, as cultural norms and societal beliefs are not easily manipulated in experimental research, thereby precluding causal inference. In the present study, infant sleep habits may be experimentally modified by asking some caregivers to place their infants in disposable diapers (PAMPERS BABY DRY®) and reduce disrupting the sleep of their infants as much as possible at night, whereas other caregivers continued using traditional cloth diapers and were not given any instruction regarding the need to minimize nighttime interactions with their infants.

The diaper manipulation produced variability in infant sleep habits. Specifically, the sleep of infants who wore cloth diapers was disrupted a greater total amount of time and a greater amount of time when accounting for disruptions that did and did not wake the infant from sleep. The sleep of infants in the cloth diaper control group was also disrupted a greater total number of times and a greater number of times when accounting for disruptions that did and did not wake the infant from sleep. Differences were not found in the amount of time infants spent sleeping after accounting for all nighttime disruptions, thereby indicating that the sleep of infants enrolled in the cloth diaper control group was more fragmented despite both groups receiving similar amounts of sleep at night.

Although the diaper manipulation was successful in producing differences in the sleeping habits of infants enrolled in the cloth diaper control and disposable diaper groups, similar temperament profiles were obtained on the majority of the subscales of the IBQ-R as assessed by between- and within-subjects analyses; correlations were more informative in indicating relations between nighttime sleep habits and temperament. Within-subjects analyses indicated that infants received marginally higher ratings on activity level at the second administration of the IBQ-R relative to the first, regardless of group assignment. Group differences in the development of temperament were apparent on the subscales of low intensity pleasure and perceptual sensitivity. Specifically, after the 6-week sleep intervention, infants who wore disposable diapers received significantly higher ratings on perceptual sensitivity, whereas infants who wore cloth control diapers received lower ratings on low intensity pleasure. These findings were also corroborated by correlations between measures of sleep and temperament near the end of the diaper manipulation. In general, the results indicated that infants with more disrupted sleep received higher ratings on activity level, approach, and distress to limitations; more disrupted sleep also related to lower scores on perceptual sensitivity and rate of recovery from distress.

The pattern of findings may be obtained from the results of the complex interplay of biologically-based culturally-invariant constraints on the development of temperament and experience-dependent processes that originate from specific social and cultural milieus. Indeed, there are many differences between Eastern and Western cultures more generally and American and Chinese cultures more specifically. The pattern of pattern of group differences may be obtained from results of caregiver interpretation of infant behavior in relation to culturally-appropriate attributes. In China, active and exploratory tendencies are often discouraged, along with aggression, impulsivity, and other surgent or extraverted behaviors. Although caregivers most commonly refrain from actively punishing or prohibiting children for these infractions until the early school years, they may still attempt to instill traditional Chinese values in infancy (Ho, 1986). In light of these cultural values, it is suggested that infants who experienced less fragmented sleep at night were rated more favorably by their caregivers relative to infants who experienced greater durations and frequencies of nighttime disruptions. That is, the temperaments of infants who had more fragmented nighttime sleep were characterized by increased activity level, increased distress when placed in limiting situations, marginally more smiling and laughter, and increased anticipation of positive activities. Notably, three of these four scales loaded onto a higher-order factor pertaining to extraversion or surgency in an American sample (Gartstein & Rothbart, 2003); some similar items also loaded onto a comparable higher-order factor in Chinese children tested on the Children's Behavior Questionnaire (CBQ; Rothbart, Ahadi, Hershey, & Fisher, 2001) (Ahadi et al., 1993).

Also observed are negative correlations between the number of nighttime sleep disruptions and perceptual sensitivity as well as decreases in low intensity pleasure over time for infants who wore cloth control diapers. These findings may be indicative of delays in cognitive functioning, including attention and language development. In contrast, less fragmented nighttime sleep related to higher ratings on rate of recovery from distress and perceptual sensitivity and resulted in higher change scores on low intensity pleasure relative to infants who experienced more fragmented sleep at night. These differences may be due to the facilitative effects of nighttime sleep on brain development (Peirano, Algarín, & Uauy, 2003) or consolidation mechanisms known to influence memory and other cognitive abilities (in infants, see Gomez, Bootzin, & Nadel, 2006; for a review, see Ellenbogen, Payne, & Stickgold, 2006). Indeed, the scale of low intensity pleasure loads onto a higher-order factor pertaining to orienting and regulation in infancy (Gartstein & Rothbart, 2003) and increases in perceptual sensitivity may be related to the development of the anterior attention system (Ruff & Rothbart, 1986). Future work is necessary to establish the validity of this possibility as well as to determine the higher-order relations underlying the development of temperament in Chinese infants.

Notably, the present study did not include investigation of the mechanism underlying the reported differences in the development of infant temperament over the 6-week diaper manipulation, but three options are plausible: (a) changes in caregiver ratings of infant temperament may have resulted from certifiable modifications of infant behavior due to the diaper manipulation, (b) caregiver perceptions of infant behavior may have been altered as a result of increased amounts of adult sleep at night due to the diaper manipulation, or (c) the reported effects could be due to their combined influence. Future research should address this question by including objective assessments of sleep and infant temperament in addition to caregiver-report measures. Indeed, the most significant shortcoming of the present work includes complete reliance on caregiver-report measures. However, the most comprehensive and objective sleep and temperament inventories available are used while asking those who knew the infants best to report on their recent behaviors. The alternatives of using activity monitors to more objectively assess patterns of sleep or coding sleep habits from videotape seemed unreasonable due to the number of infants tested, the duration of the diaper manipulation, and the preliminary nature of the investigation. In addition, obtaining objective ratings of infant temperament were also not possible due to the extensive test battery in which infants participated. In the future, inclusion of objective ratings of infant temperament may yield higher rates of stability and a greater number of significant findings than those reported, although measures of internal consistency in the reported range are not uncommon in studies of Chinese infants tested with the IBQ (Gartstein et al., 2006) or children tested with the CBQ (Ahadi et al., 1993). Nevertheless, the limitations of this work are far outweighed by its contribution to the literature: the reported experiment is one of few designed to investigate the influence of a specific cultural factor on the development of temperament in infancy.

In conclusion, the reported findings contribute to the temperament literature by indicating that sleep habits influence the development of temperament over a six-week sleep intervention. They also contribute to the sleep literature by indicating that normative sleep habits may influence the development of temperament. Such findings are notable, in that research has not yet indicated a causal relation between sleep and temperament: most previous studies have included investigation of naturalistic relations between sleep and temperament instead of manipulating sleep habits to determine their influence on behavior. In addition, many studies of relations between sleep and temperament have not been conducted with normative samples: most have included investigation of sleep problems in relation to temperament outcomes (Carey, 1974; Sadeh, Lavie, & Scher, 1994; Scher, Tirosh, & Lavie, 1998; Weissbluth et al., 1984; Weissbluth & Liu, 1983). The present research indicates that, in addition to other factors, sleep habits may contribute to differences in the development of temperament both within and across cultures. Future research should be conducted to test for replication of the reported pattern of results, to elucidate the mechanism responsible for the observed differences in temperament, and to determine relations between sleep and temperament in infancy and socioemotional outcomes over the long term.

It will be appreciated that any of the features, actions, or aspects of the present technical disclosure described herein may be combined, in whole or part, with any other feature, action, or aspect.

Some embodiments of this disclosure gather, store, and present data used to evaluate if and how disposable diapers might help to reduce fragmentation of infant nocturnal sleep, such as sleep fragmentation that is associated with cloth diaper practices. Some embodiments of this disclosure gather, store, and present data used to provide a first available systematic assessment of sleep quality among infants, in the context of diaper care practices. Further, some embodiments of this disclosure gather, store, and present data used to provide a first available systematic assessment of sleep quality based on diaper wear and developmental indicators. Machines and processes including some of the embodiments described herein that analyze the relationship of diaper wear with sleep and/or developmental indicators help to promote the development of diaper products and promote the research of the relationship between diaper wear with sleep and/or developmental indicators. Some embodiments of the present disclosure provide a machine, manufacture, and process for analyzing a relationship between diaper wear and sleep and/or developmental indicators.

Conventional sleep monitoring devices and procedures have been administered in analyzing people who have been diagnosed with sleep disorders. Conventional sleep monitoring devices also have been administered in analyzing the relationship between normal sleep, this is to say, sleep where no evidence of disorder or pathology is present and the normal function and performance of the human subject, such as cognition, memory, physical performance and endurance, etc. Besides conventional sleep monitoring devices and procedures using sleep monitoring devices as stated above, conventional sleep monitoring procedures have been used to find relationships between sleep and temperament, without any information on causation and without any information on diaper wear. Moreover, it has never been inferred, in a scientifically proven way that a relationship may exist between diaper quality and/or performance and/or the quality of sleep a child may achieve while using a diaper product and/or that this relation may have relevant implications for the child's mental performance and/or development. Embodiments of the present disclosure provide a machine, manufacture, and process for analyzing a relationship between diaper wear and sleep and/or developmental indicators as disclosed in the claims.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An analysis machine configured to facilitate analysis of the effect of diaper wear on sleep and/or developmental indicators comprising:
a disposable diaper verification module configured to accept verification input indicating that a disposable diaper was secured on at least one of a first plurality of subjects;
a sleep initiation monitoring module configured to monitor the subject for sleep initiation data;
a sleep evaluation module configured to evaluate the sleep of the subject based upon sleep evaluation data.

2. The machine according to claim 1, further comprising
a developmental indicator input module configured to accept data of a developmental indicator of the subject of the first plurality, and
a display module configured to display the developmental indicator data or the sleep evaluation data of the subject of the first plurality,
wherein the developmental indicator data and the sleep evaluation data are collected during a coinciding time duration.

3. The machine according to claim 2, further comprising
a cloth diaper verification module configured to accept verification input indicating that a cloth diaper was secured to at least one of a second plurality of subjects;
a sleep initiation monitoring module configured to monitor the subject of the second plurality for sleep initiation data;
a sleep evaluation module configured to evaluate the sleep of the subject of the second plurality based upon sleep evaluation data;
a developmental indicator input module configured to accept data of a developmental indicator of the subject of the second plurality, and
a display module configured to display the developmental indicator data or the sleep evaluation data of the subject of the second plurality,
wherein the developmental indicator data and the sleep evaluation data of the subject of the second plurality are collected during a coinciding time duration.

4. The machine according to claim 3, further comprising
a comparator module configured to compare the developmental indicator data of the subject of the first plurality and the developmental indicator data of the subject of the second plurality.

5. The machine according to claim 2, further comprising
a comparator module configured to compare data of the sleep of the subject of the first plurality and the sleep of the subject of the second plurality.

6. The machine according to claim 1, wherein the sleep initiation monitoring module or the sleep evaluation module is any one or more of a group consisting of: a polysomnography device, an actigraphy device, or a video monitoring device, or a remote sensing device configured to utilize Doppler radiofrequency for remote sensing of physical activity and respiratory rate, or software configured to enable processing of a physical motion signal and/or a respiratory signal, or a computational algorithm configured to enable processing of a physical motion signal and/or a respiratory signal, or a remote sensing device configured to determine a sleep/wake status of the subject, or software and/or computational algorithm configured to enable user-dictated modification or modulation of the software and/or algorithm parameters that affect the determination of a sleep/wake status based on activity and/or respiratory rate data.

7. The machine according to claim 6, wherein the polysomnography module is any one or more of a group consisting of: an electroencephalogram (EEG), an electrooculogram (EOG), an electromyogram (EMG), an electrocardiogram (ECG), strain gauges, a pressure transducer, a thermocouple, a breathing monitor, a respiratory airflow monitor, a respiratory effort indicators, a peripheral pulse oximetry device, a piezoelectric belt, an inductive plethysmography device, an impedance pneumography device, an inductance pneumography device, an endoesophageal pressure device, a thermistor, a pneumotachograph, a pulse oximetry device, a body position monitor, a mercury switch, a vibration recorder, an end-tidal C02 device, a transcutaneous C02 device, an esophageal pH device, a penile tumescence device, or a bipolar EEG device.

8. The machine according to claim 6, wherein the actigraphy module is any one or more of the grouping consisting of: a zero crossing computing device, a proportional integral computing device, an area under the curve computing device, a time above threshold computing device, or a length of time that the wave is above a certain threshold computing device.

9. The machine according to claim 6, wherein the sleep evaluation module includes sleep diary recorded data.

10. The machine according to claim 6, wherein the sleep evaluation module includes Infant Behavior Questionnaire-Revised (IBQ-R) data.

11. The machine according to claim 6, wherein the sleep evaluation module includes Infant Behavior Questionnaire-Revised (IBQ-R) data based on a 7-point Likert-type scale.

12. The machine according to claim 6, wherein the sleep evaluation module includes a test duration and one or more time specified evaluation, wherein the one or more time specified evaluation is taken prior to the end of the test duration.

13. The machine according to claim 6, wherein the developmental indicator data and the sleep evaluation data are collected during a coinciding time duration.

14. The machine according to claim 6, wherein the sleep evaluation device includes duration and frequency data of sleep including data of uninterrupted sleep duration.

15. The machine according to claim 6, wherein the sleep evaluation device includes duration or frequency data of sleep including any one or more of the following: a cumulative number of times the subject was disturbed, a number of times the subject was disturbed and woken from sleep, or a number of times the subject was disturbed but not woken, wherein a mean value is created based on an average value across data from three consecutive days.

16. The machine according to claim 1, wherein the sleep evaluation device generates scales based on an Infant Behavior Questionnaire-Revised (IBQ-R) data.

17. The machine according to claim 16, wherein the scales are any one or more of the group consisting of: activity level data, approach data, cuddliness data, distress to limitations data, duration of orienting data, fear data, high-intensity pleasure data, low intensity pleasure data, perceptual sensitivity data, rate of recovery from distress data, sadness data, smiling and laughter data, soothability data, or vocal reactivity data.

18. An analysis machine configured to facilitate analysis of the effect of diaper wear on developmental indicators comprising:
a disposable diaper verification module configured to accept verification input indicating that a disposable diaper was secured on at least one of a first plurality of subjects;
a sleep initiation monitoring module configured to monitor the subject for sleep initiation data;
a sleep evaluation module configured to evaluate the sleep of the subject based upon sleep evaluation data;
a developmental indicator input module configured to accept data of a developmental indicator of the subject of the first plurality, and
a display module configured to display the developmental indicator data or the sleep evaluation data of the subject of the first plurality,
a cloth diaper verification module configured to accept verification input indicating that a cloth diaper was secured to at least one of a second plurality of subjects;
a sleep initiation monitoring module configured to monitor the subject of the second plurality for sleep initiation data;
a sleep evaluation module configured to evaluate the sleep of the subject of the second plurality based upon sleep evaluation data;
a developmental indicator input module configured to accept data of a developmental indicator of the subject of the second plurality, and
a display module configured to display the developmental indicator data or the sleep evaluation data of the subject of the second plurality,
wherein the developmental indicator data and the sleep evaluation data of the subject of the first plurality are collected during a coinciding time duration,
wherein the developmental indicator data and the sleep evaluation data of the subject of the second plurality are collected during a coinciding time duration;
a comparator module configured to compare the developmental indicator data of the subject of the first plurality and the developmental indicator data of the subject of the second plurality;
a comparator module configured to compare data of the sleep of the subject of the first plurality and the sleep of the subject of the second plurality,
wherein the sleep initiation monitoring module is any one or more of a group consisting of: a polysomnography device, an actigraphy device, or a video monitoring device, wherein the actigraphy module is any one or more of the grouping consisting of: a zero crossing computing device, a proportional integral computing device, an area under the curve computing device, a time above threshold computing device, or a length of time that the wave is above a certain threshold computing device.

19. A computer program product having a computer readable medium including programmed instructions for facilitating analysis of the effect of diaper wear on sleep and/or developmental indicators, wherein the instructions, when executed by a processor configured to facilitate the analysis of the effect of diaper wear on the sleep and/or the developmental indicators causes the processor to perform:
verifying a disposable diaper was secured on at least one of a first plurality of subjects;
monitoring the subject for sleep initiation data;
evaluating the sleep of the subject based upon sleep evaluation data.

20. The computer program product according to claim 19, further comprising
accepting data of a developmental indicator of the subject of the first plurality, and
displaying the developmental indicator data or the sleep evaluation data of the subject of the first plurality,
wherein the developmental indicator data and the sleep evaluation data are collected during a coinciding time duration.

21. The computer program product according to claim 20, further comprising
verifying that a cloth diaper was secured to at least one of a second plurality of subjects;
monitoring the subject of the second plurality for sleep initiation data;
evaluating the sleep of the subject of the second plurality based upon sleep evaluation data;
accepting data of a developmental indicator of the subject of the second plurality, and
displaying the developmental indicator data or the sleep evaluation data of the subject of the second plurality,
wherein the developmental indicator data and the sleep evaluation data of the subject of the second plurality are collected during a coinciding time duration.

22. The computer program product according to claim 21, further comprising
comparing the developmental indicator data of the subject of the first plurality and the developmental indicator data of the subject of the second plurality.

23. The computer program product according to claim 20, further comprising
comparing data of the sleep of the subject of the first plurality and the sleep of the subject of the second plurality.

24. The computer program product according to claim 19, wherein the sleep initiation monitoring or the sleep evaluating includes using any one or more of a group consisting of: a polysomnography device, an actigraphy device, or a video monitoring device, or a remote sensing device configured to utilize Doppler radiofrequency for remote sensing of physical activity and respiratory rate, or software configured to enable processing of a physical motion signal and/or a respiratory signal, or a computational algorithm configured to enable processing of a physical motion signal and/or a respiratory signal, or a remote sensing device configured to determine a sleep/wake status of the subject, or software and/or computational algorithm configured to enable user-dictated modification or modulation of the software and/or algorithm parameters that affect the determination of a sleep/wake status based on activity and/or respiratory rate data.

25. The computer program product according to claim 24, wherein the polysomnography module is any one or more of a group consisting of: an electroencephalogram (EEG), an electrooculogram (EOG), an electromyogram (EMG), an electrocardiogram (ECG), strain gauges, a pressure transducer, a thermocouple, a breathing monitor, a respiratory airflow monitor, a respiratory effort indicators, a peripheral pulse oximetry device, a piezoelectric belt, an inductive plethysmography device, an impedance pneumography device, an inductance pneumography device, an endoesophageal pressure device, a thermistor, a pneumotachograph, a pulse oximetry device, a body position monitor, a mercury switch, a vibration recorder, an end-tidal C02 device, a transcutaneous C02 device, an esophageal pH device, a penile tumescence device, or a bipolar EEG device.

26. The computer program product according to claim 24, wherein the actigraphy module is any one or more of the grouping consisting of: a zero crossing computing device, a proportional integral computing device, an area under the curve computing device, a time above threshold computing device, or a length of time that the wave is above a certain threshold computing device.

27. The computer program product according to claim 24, wherein the sleep evaluation module includes sleep diary recorded data.

28. The computer program product according to claim 24, wherein the sleep evaluation module includes Infant Behavior Questionnaire-Revised (IBQ-R) data.

29. The computer program product according to claim 24, wherein the sleep evaluation module includes Infant Behavior Questionnaire-Revised (IBQ-R) data based on a 7-point Likert-type scale.

30. The computer program product according to claim 24, wherein the sleep evaluation module includes a test duration and one or more time specified evaluation, wherein the one or more time specified evaluation is taken prior to the end of the test duration

31. The computer program product according to claim 24, wherein the developmental indicator data and the sleep evaluation data are collected during a coinciding time duration.

32. The computer program product according to claim 24, wherein the sleep evaluation device includes duration and frequency data of sleep including data of uninterrupted sleep duration.

33. The computer program product according to claim 24, wherein the sleep evaluation device includes duration or frequency data of sleep including any one or more of the following: a cumulative number of times the subject was disturbed, a number of times the subject was disturbed and woken from sleep, or a number of times the subject was disturbed but not woken, wherein a mean value is created based on an average value across data from three consecutive days.

34. The computer program product according to claim 19, wherein the sleep evaluation device generates scales based on an Infant Behavior Questionnaire-Revised (IBQ-R) data.

35. The computer program product according to claim 34, wherein the scales are any one or more of the group consisting of: activity level data, approach data, cuddliness data, distress to limitations data, duration of orienting data, fear data, high-intensity pleasure data, low intensity pleasure data, perceptual sensitivity data, rate of recovery from distress data, sadness data, smiling and laughter data, soothability data, or vocal reactivity data.

36. A process for facilitating analysis of the effect of diaper wear on sleep and/or developmental indicators, the process comprising:
verifying a disposable diaper was secured on at least one of a first plurality of subjects;
monitoring the subject for sleep initiation data;
evaluating the sleep of the subject based upon sleep evaluation data via a sleep evaluation module.

37. The process according to claim 36, further comprising
accepting data of a developmental indicator of the subject of the first plurality, and
displaying the developmental indicator data or the sleep evaluation data of the subject of the first plurality,
wherein the developmental indicator data and the sleep evaluation data are collected during a coinciding time duration.

38. The process according to claim 37, further comprising
verifying that a cloth diaper was secured to at least one of a second plurality of subjects;
monitoring the subject of the second plurality for sleep initiation data;
evaluating the sleep of the subject of the second plurality based upon sleep evaluation data;
accepting data of a developmental indicator of the subject of the second plurality, and
displaying the developmental indicator data or the sleep evaluation data of the subject of the second plurality,
wherein the developmental indicator data and the sleep evaluation data of the subject of the second plurality are collected during a coinciding time duration.

39. The process according to claim 38, further comprising
comparing the developmental indicator data of the subject of the first plurality and the developmental indicator data of the subject of the second plurality.

40. The process according to claim 37, further comprising
comparing data of the sleep of the subject of the first plurality and the sleep of the subject of the second plurality.

41. The process according to claim 36, wherein the sleep initiation monitoring or the sleep evaluating includes using any one or more of a group consisting of: a polysomnography device, an actigraphy device, or a video monitoring device, or a remote sensing device configured to utilize Doppler radiofrequency for remote sensing of physical activity and respiratory rate, or software configured to enable processing of a physical motion signal and/or a respiratory signal, or a computational algorithm configured to enable processing of a physical motion signal and/or a respiratory signal, or a remote sensing device configured to determine a sleep/wake status of the subject, or software and/or computational algorithm configured to enable user-dictated modification or modulation of the software and/or algorithm parameters that affect the determination of a sleep/wake status based on activity and/or respiratory rate data.

42. The process according to claim 41, wherein the polysomnography module is any one or more of a group consisting of: an electroencephalogram (EEG), an electrooculogram (EOG), an electromyogram (EMG), an electrocardiogram (ECG), strain gauges, a pressure transducer, a thermocouple, a breathing monitor, a respiratory airflow monitor, a respiratory effort indicators, a peripheral pulse oximetry device, a piezoelectric belt, an inductive plethysmography device, an impedance pneumography device, an inductance pneumography device, an endoesophageal pressure device, a thermistor, a pneumotachograph, a pulse oximetry device, a body position monitor, a mercury switch, a vibration recorder, an end-tidal C02 device, a transcutaneous C02 device, an esophageal pH device, a penile tumescence device, or a bipolar EEG device.

43. The process according to claim 41, wherein the actigraphy module is any one or more of the grouping consisting of: a zero crossing computing device, a proportional integral computing device, an area under the curve computing device, a time above threshold computing device, or a length of time that the wave is above a certain threshold computing device.

44. The process according to claim 41, wherein the sleep evaluation module includes sleep diary recorded data.

45. The process according to claim 41, wherein the sleep evaluation module includes Infant Behavior Questionnaire-Revised (IBQ-R) data.

46. The process according to claim 41, wherein the sleep evaluation module includes Infant Behavior Questionnaire-Revised (IBQ-R) data based on a 7-point Likert-type scale.

47. The process according to claim 41, wherein the sleep evaluation module includes a test duration and one or more time specified evaluation, wherein the one or more time specified evaluation is taken prior to the end of the test duration.

48. The process according to claim 41, wherein the developmental indicator data and the sleep evaluation data are collected during a coinciding time duration.

49. The process according to claim 41, wherein the sleep evaluation device includes duration and frequency data of sleep including data of uninterrupted sleep duration.

50. The process according to claim 41, wherein the sleep evaluation device includes duration or frequency data of sleep including any one or more of the following: a cumulative number of times the subject was disturbed, a number of times the subject was disturbed and woken from sleep, or a number of times the subject was disturbed but not woken, wherein a mean value is created based on an average value across data from three consecutive days.

51. The process according to claim 36, wherein the sleep evaluation device generates scales based on an Infant Behavior Questionnaire-Revised (IBQ-R) data.

52. The process according to claim 51, wherein the scales are any one or more of the group consisting of: activity level data, approach data, cuddliness data, distress to limitations data, duration of orienting data, fear data, high-intensity pleasure data, low intensity pleasure data, perceptual sensitivity data, rate of recovery from distress data, sadness data, smiling and laughter data, soothability data, or vocal reactivity data.
